SEQUENCE LISTING (9 sequences)

SEQ ID NO: 1 - DNA, 23 bp, artificial sequence
  synthetic oligonucleotide
agcaccacactcacaccctcttt

SEQ ID NO: 2 - DNA, 24 bp, artificial sequence
  synthetic oligonucleotide
tccttgattttctccttgatggcc

SEQ ID NO: 3 - DNA, 24 bp, artificial sequence
  synthetic oligonucleotide
tccctgtcagagaaaacctggttt

SEQ ID NO: 4 - DNA, 24 bp, artificial sequence
  synthetic oligonucleotide
acatctactatgtgtacagtttcc

SEQ ID NO: 5 - DNA, 25 bp, artificial sequence
  synthetic oligonucleotide
aaaaaccacggtgcggccttgggta

SEQ ID NO: 6 - DNA, 19 bp, artificial sequence
  synthetic oligonucleotide
ccacggtgctgcagggcat

SEQ ID NO: 7 - DNA, 19 bp, artificial sequence
  synthetic oligonucleotide
tgaccaagatgctggagat

SEQ ID NO: 8 - DNA, 19 bp, artificial sequence
  synthetic oligonucleotide
cggaatgggagcacagtca

SEQ ID NO: 9 - DNA, 19 bp, artificial sequence
  synthetic oligonucleotide
gcacagtcatcgcagatga

(19) United States
(12) Patent Application Publication
(10) Pub. No.: US 2008/0045443 A1
(43) Pub. Date: Feb. 21, 2008

(54) Compositions for Inhibiting plexin-A1-DAP12 interactions and Methods of Treating Inflammatory, Autoimmune or Bone Resorption Diseases Using the Same

(76) Inventors: Hitoshi KIKUTANI, Osaka (JP); Atsushi Kumanogoh, Osaka (JP); Kenji Sugiyama, Amagasaki (JP)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(21) Appl. No.: 11/550,166

(22) Filed: Oct. 17, 2006

(30) Foreign Application Priority Data
May 11, 2006 (DE) 20 2006 007 499

(51) Int. Cl. A61K 38/17; A61P 19/00; A61P 29/00; A61P 37/00; C07K 16/18; C12Q 1/68; G01N 33/68

(52) U.S. Cl. 514/2; 435/29; 435/6; 530/389.1

(57) ABSTRACT

Disclosed are compositions and methods of treating an inflammatory, autoimmune or bone resorption disease by inhibiting plexin-A1-DAP12 interaction, plexin-A1-Trem-2 interaction or DAP12-Trem-2 interaction.

This application claims benefit to German Application DE 20 2006 007 499.6 filed May 11, 2006.

### BACKGROUND OF THE INVENTION

1. Technical Field

This invention relates to compositions and methods of treating an inflammatory, autoimmune or bone resorption disease by inhibiting plexin-A1-DAP12 interaction, plexin-A1-Trem-2 interaction or DAP12-Trem-2 interaction.

2. Background Information

Semaphorins and their receptors play diverse roles in axon guidance, organogenesis, vascularization/angiogenesis, oncogenesis, and regulation of immune responses¹⁻¹¹. The primary receptors for semaphorins are members of the plexin family^{2,12-14}. In particular, plexin-A1, with ligand-binding neuropilins, transduces repulsive axon guidance signals for soluble class III semaphorins¹⁵, whereas plexin-A1 plays multiple roles in chick cardiogenesis as a receptor for a transmembrane semaphorin Sema6D independent of neuropilins¹⁶. Additionally, plexin-A1 has been implicated in dendritic cell (DC) functions in the immune system¹⁷. However, the role of plexin-A1 in vivo and its important roles in immune responses and in bone homeostasis up until the present invention have been unclear.

### BRIEF SUMMARY OF THE INVENTION

It is therefore an object of the invention to provide a method of treating an inflammatory, autoimmune or bone resorption disease, by administering to a patient a composition which inhibits plexin-A1-DAP12 interaction.

It is a further object of the invention to provide a method of treating an inflammatory, autoimmune or bone resorption disease, by administering to a patient a composition which inhibits plexin-A1-Trem-2 interaction.

It is yet another object of the invention to provide a method of treating an inflammatory, autoimmune or bone resorption disease, by administering to a patient a composition which inhibits DAP12-Trem-2 interaction.

It is yet still another object of the invention to provide a method to identify a compound that controls interaction of plexin-A1 with DAP12 activity in a cell, comprising: (1) contacting a cell with a putative regulatory compound, wherein the cell includes a plexin-A1 protein and a DAP12 protein; and (2) assessing the ability of the putative regulatory compound to inhibit the interaction of plexin-A1 with DAP12.

It is yet still another object of the invention to provide a composition that controls interaction of plexin-A1 with DAP12 activity in a cell wherein the composition is therapeutically useful in treating an inflammatory, autoimmune or bone resorption disease.

### BRIEF DESCRIPTION OF THE FIGURES

FIG. 1: Generation of plexin-A1−/− mice. (a) Disruption of the plexin-A1 gene. The gene structure of wild-type plexin-A1 allele (top), plexin-A1-targeting construct (middle) and the resulting plexin-A1 mutant allele (bottom) are shown. Filled boxes denote exons. The 3.0-kb fragment containing the initiation codon and the coding sequence of the sema-domain was replaced with Neo. The HSV-tk gene was appended to allow for selection against random integration. B, BamHI. (b) Southern blot analysis. To assess the genotype of wild-type (+/+), heterozygous (±), and homozygous (−/−) mutant mice, tail DNA was digested with BamHI, electrophoresed, and hybridized with the radio-labelled probe that is shown by a gray box in (a). The 11.5-kb fragment represents the wild-type allele, and the 2.5-kb fragment depicts the targeted allele. (c) Northern blot analysis. RNA prepared from the brain of wild-type (+/+) or plexin-A1−/− (−/−) mice was electrophoresed, and hybridized with radio-labelled probes. (d) RT-PCR analysis. cDNAs derived from the brain, heart and spleen of wild-type (+/+) or plexin-A1−/− (−/−) mice were used for RT-PCR to determine plexin-A1 expression.

FIG. 2: There are no apparent differences between wild-type (+/+) and plexin-A1−/− (−/−) embryos (E12). Whole-mount staining of wild-type (+/+) or plexin-A1−/− (−/−) E12 embryos using anti-neurofilament antibodies (2H3) as described previously (Giger et al, Neuron 25:29, 2000). IV, trochlear nerve; V, trigeminal nerve; Vop, opthalmic branch of the trigeminal nerve; Vmax, maxillary branch of trigeminal nerve; VII, facial nerve; VIII, vestibulocochlear nerve; X, vague nerve.

FIG. 3: Expression profile of plexin-A1. Expression of plexin-A1 transcripts was determined by RT-PCR using multiple mouse tissue panel cDNAs (Clontech). G3PDH-transcripts were used as controls.

FIG. 4: Normal development of lymphocytes in plexin-A1−/− mice. Cells were prepared from the thymus and spleen of wild-type (+/+) or plexin-A1−/− (−/−) mice, stained with various antibodies and analysed by flow cytometry.

FIG. 5: Normal B-cell proliferative responses in plexin-A1−/− mice. Small resting B-cells prepared from wild-type (open circles) or plexin-A1−/− mice (closed circles) were cultured for 72 h with various concentrations of the indicated factors. [3H]-thymidine was added for the last 14 h. Data are the mean±S.D.

FIG. 6: Normal CD4+ T-cell proliferative responses in plexin-A1−/− mice. CD4+ T-cells purified from wild-type (open circles) or plexin-A1−/− mice (closed circles) were cultured with various concentrations of immobilized anti-CD3 in the absence (a) or presence (b) of anti-CD28 (10 μg ml-1) for 48 h. [3H]-thymidine was added for the last 14 h. Data are the mean±S.D.

FIG. 7: Normal osteoblast functions in plexin-A1−/− mice. (a, b) Comparable expression of osteocalcin (a) and BSP (b) in plexin-A1−/− osteoblasts. The levels of serum osteocalcin were determined by ELISA. Expression of transcripts of bone sialoprotein (BSP) in wild-type (+/+) or plexin-A1−/− (−/−) osteoblasts was determined by real time monitoring quantitative PCR analysis. G3PDH-transcripts were used as internal controls. Data are the mean±S.D. (c) A typical calcein experiment is shown. Mineral apposition rate (MAR) and bone formation rate (BFR) of wild-type (+/+) and plexin-A1−/− (−/−) mice are shown. Data are from 3 mice±s.e.m.

FIG. 8: Expression profiles of Sema6D transcripts in T-cells and osteoclasts. (a) cDNA was prepared from unstimulated- or anti-CD3 stimulated-T-cells. Expression of transcripts of Sema6D was determined by real time monitoring quantitative PCR analysis. G3PDH-transcripts were used as internal controls. (b) cDNA was prepared from resting T-cells (−), Th1 (IL-12 plus anti-IL-4)- and Th2 (IL-4 plus anti-IL-12)-polarized cells. Expression of transcripts of Sema6D and G3PDH in these cells was determined by PCR using their specific primers. cDNA was also prepared from osteoclasts induced by M-CSF plus RANKL (in vitro) or from femurs and tibiae of 13-day-old mice (primary). Expression of transcripts of plexin-A1 and G3PDH were determined by PCR using their specific primers.

FIG. 9: Control soluble Sema4A proteins exert no effects on DCs and osteoclasts. (a) Recombinant Sema4A does not bind to DCs. Wild-type BMDCs were cultured with anti-CD40 mAb for 24 h and stained with biotinylated recombinant Sema4A (thick lines) or biotinylated human IgG1 (dotted lines) plus streptavidin-APC. (b) Sema4A does not induce IL-12 production by DCs. BMDCs from wild-type mice were cultured with recombinant Sema4A, Sema6D-Fc, anti-CD40 mAbs or LPS for 72 h and production of IL-12p40 was measured by ELISA. Data are the mean±S.D. (c) Sema4A has no effects on osteoclast development. Bone marrow cells from wild-type mice were cultured with M-CSF (10 ng ml-1) and Sema4A-Fc (10 μg ml-1) for 2 days, and further cultured with M-CSF (10 ng ml-1) and a suboptimal dose of RANKL (5 ng ml-1) for 3 days. The number of TRAP-positive cells was measured.

FIG. 10: Reduced but substantial Sema6D binding observed in plexin-A1−/− preosteoclasts. Osteoclast precursor cells from wild-type (+/+) or plexin-A1−/− (−/−) littermates were stained with biotinylated Sema6D-Fc (thick lines) or biotinylated human IgG1 (dotted lines) plus streptavidin-APC.

FIG. 11: Expression profiles of plexin-A subfamily members in DCs. cDNA was prepared from BMDCs stimulated with anti-CD40. Expression of transcripts of plexin-A1, -A2, -A3 -A4 and G3PDH were determined by PCR using their specific primers. As controls, 10 ng of cDNAs of plexin-A1, -A2, -A3 and -A4 were amplified by PCR by their specific primers.

FIG. 12: Fluorescence resonance energy transfer (FRET) between Plexin-A1 and Trem-2. The binding of PlexinA1 (deleted in its cytoplasmic region)-CFP to Trem-2-YFP or YFP-pm (control) was analyzed in COS7 cells by intermolecular FRET. COS7 cells were transfected with expression plasmids and imaged for YFP, FRET, and CFP, which were used to represent FRETC in the IMD mode. Eight colors (red to blue) represent FRET efficiency, whereby the intensity of each color indicates the mean intensity CFP. The scale bar shows 10 μm. pCAGGS-YFP-pm encoded a YFP fused to the carboxy-terminus of K-Ras4B protein (a.a. 169-188).

(Intermolecular FRET analysis) COS7 cells expressing the fluorescent probes were imaged and the data were processed as described previously (a). In brief, fluorescent images were acquired sequentially through YFP (excitation, 510/23 nm; emission, 560/15 nm), CFP (excitation, 420/20 nm; emission, 480/20 nm), and FRET (excitation, 420/20 nm; emission, 535/35 nm) filter channels. Fluorescence through the FRET filter set consisted of a FRET-component (“corrected” FRET, FRETC) and non-FRET components, spectral bleedthrough and cross-excitation. The non-FRET components were subtracted as previously described (b). For our experimental conditions, we used the following equation:
FRETC=FRET−(0.34×CFP)−(0.10×YFP)

After the calculation of FRETC, statistical analysis was performed with Microsoft Excel. a. Terai, K. & Matsuda, M. Ras binding opens c-Raf to expose the docking site for mitogen-activated protein kinase kinase. EMBO Rep. 6, 251-255 (2005). b. Sorkin, A., McClure, M., Huang, F. & Carter, R. Interaction of EGF receptor and grb2 in living cells visualized by fluorescence resonance energy transfer (FRET) microscopy. Curr. Biol. 10, 1395-1398 (2000).

FIG. 13: Plexin-A1−/− mice were resistant to EAE induced by immunization with a MOG-peptide. (a) 6-8-wk-old wild-type (n=8) and plexin-A1−/− (n=8) mice were immunized with 100 μg of MOG 35-55 in CFA subcutaneously. 100 ng of pertussis toxin was injected intravenously on the day of immunization and 2 d later. The mice were clinically scored daily: 0, no disease; 1, limp tail; 2, hind limb weakness; 3, hind limb paralysis; 4, hind and forelimb paralysis; 5, moribund state. Mean clinical score was calculated by averaging the scores of all mice, including animals that did not develop EAE. (b) The spinal cord of plexin-A1−/− mice were not infiltrated with inflammatory mononucletic cells. Spinal cords were removed and fixed in 10% formalin. Paraffin-embedded sections were stained with hematoxylin-eosin for light microscopy.

(c) Impaired T-cell priming in plexin-A1−/− mice. Wild-type (open circles) and plexin-A1−/− mice (closed circles) were immunized with 100 μg of MOG 35-55 in CFA into the hind footpad. Seven days after priming, cells prepared from the draining lymph nodes were re-stimulated with various concentrations of MOG 35-55.

FIG. 14: Impaired calcium oscillation in plexin-A1−/− cells. Calcium signalling in plexin-A1−/− osteoclast precursor cells stimulated with M-CSF and RANKL. Osteoclast precursor cells from wild-type (+/+) or plexin-A1−/− (−/−) mice were incubated with RANKL in the presence of M-CSF for 24 h and subjected to calcium measurement as previously described 25.

FIG. 15: Activation of Rac induced by Sema6D is not affected by the absence of DAP12. Wild-type (+/+) or DAP12−/− (−/−) BMDCs were stimulated with Sema6D-Fc or control IgG for 30 min. Cell lysates were incubated with PAK-1-GST-agarose or Protein-G sepharose plus anti-Rac mAb, and blotted with anti-Rac mAb.

FIG. 16: (a-b) FITC in plexin-A1−/− mice in comparison to those seen in wild-type littermates and (c) expression levels of co-stimulatory molecules, including CD40, CD80, CD86 and MHC class II, between wild-type and plexin-A1−/− DCs.

FIG. 17: (a) plexin-A1−/− DCs stimulated allogeneic T-cells compared to wild-type DCs. (b) cell proliferation of CD4+ T-cells from plexin-A1−/− mice or wild-type littermates cultured with allogeneic wild-type DCs. (c) ability of plexin-A1−/− DCs to stimulate antigen-specific T cells in vitro. (d) Proliferative responses and cytokine production by CD4+ T-cells in plexin-A1−/− mice.

FIG. 18: (a-b) bone mass from three-dimensional microstructural analyses using high-resolution microcomputed tomography. (c) bone morphometric analyses. (d) sections of plexin-A1−/− long bones showing increased trabecular mass compared to wild-type bones. (e) plexin-A1−/− osteoblasts promoted the formation of wild-type osteoclasts to the same extent as wild-type osteoblasts. (f) bone morphometric analysis showed normal ratios of osteoblast surface to bone surface.

FIG. 19: (a) histological bone morphometric analyses using TRAP-staining showed that plexin-A1−/− mice showing decreased osteoclast numbers and lower ratios of osteoclast surface to bone surface. (b) plexin-A1−/− mice displayed a decrease in deoxypirydinoline (Dpyd) and collagen type I fragments. (c) in vitro induction of osteoclasts 18,19 was reduced in the absence of plexin-A1. (d) plexin-A1 was predominantly expressed in freshly isolated osteoclasts.

FIG. 20: (a-c) incubation of DCs with recombinant soluble Sema6D 16 induced IL-12 production and the up-regulation of MHC class II-expression. (d) soluble recombinant Sema6D promoted substantial osteoclast differentiation in vitro.

FIG. 21: (a) screen of several candidate molecules with putative functions in both DCs and osteoclasts for association with plexin-A1. (b and c) association of plexin-A1 with DAP12 in the presence of Trem-2. (d and e) cell expression of these plexin-A1 with DAP12.

FIG. 22: (a-b) ‘loss of function’ experiment to determine if RNAi against Trem-2 reduced the stimulatory activities of Sema6D on DCs (FIG. 22b). (c) DAP12−/− DCs exhibited considerably reduced responses to Sema6D. (d) RAW264.7 cells expressing plexin-A1, Trem-2 and DAP12 were stimulated with recombinant soluble Sema6D protein, tyrosine phosphorylation of DAP12 was observed.

### DETAILED DESCRIPTION OF THE INVENTION

In a first generic embodiment, there is provided a method of treating an inflammatory, autoimmune or bone resorption disease, by administering to a patient a composition which inhibits plexin-A1-DAP12 interaction.

The present inventors generated plexin-A1-deficient (plexin-A1−/−) mice and identified its important roles not only in immune responses but also in bone homeostasis.

Furthermore, we show that plexin-A1 associates with the triggering receptor expressed on myeloid cells-2 (Trem-2), linking semaphorin-signaling to the immuno-receptor tyrosine-based activation motif (ITAM)-bearing adaptor protein, DAP12. Thus, these findings reveal an unexpected role for plexin-A1 and present a novel signaling mechanism for exerting pleiotropic functions of semaphorins.

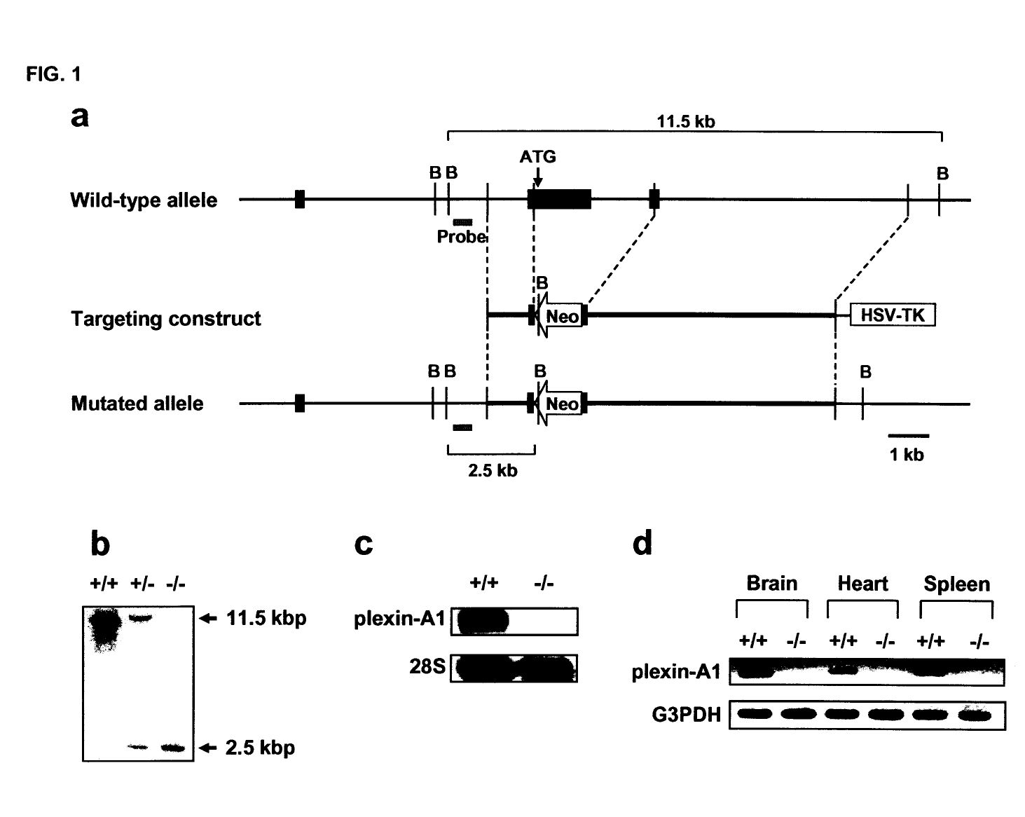

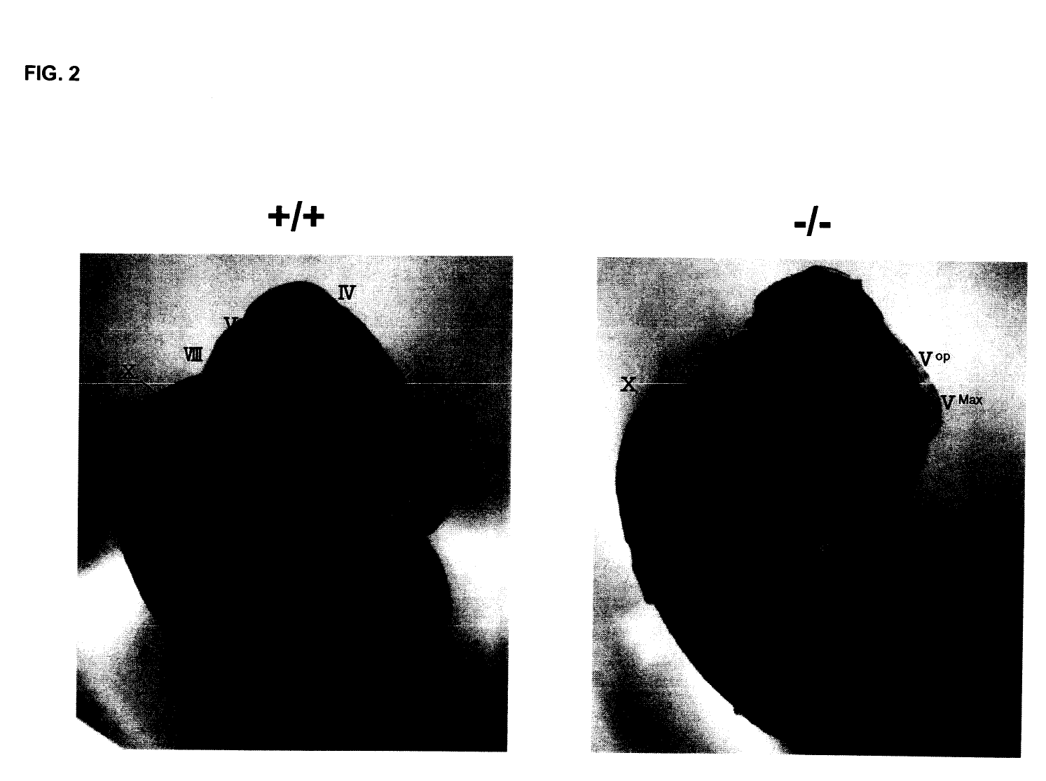

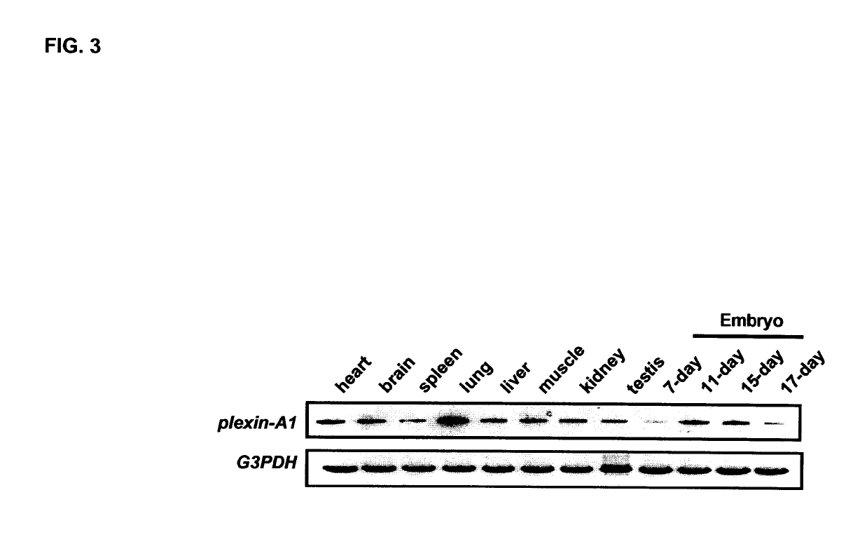

In order to better understand the role of plexin-A1 in vivo, the present inventors generated mice deficient in the plexin-A1 gene by homologus recombination by gene targeting (see FIG. 1), and we confirmed the successful deletion of plexin-A1 by both Northern blotting and reverse transcription polymerase chain reaction (RT-PCR) (see FIG. 1). Mice were born with the expected Mendelian ratios from intercrosses of heterozygous mutants, and the resulting plexin-A1−/− mice were fertile. Apparent abnormalities were not observed by gross macroscopic or histological examination of the embryos (E11.5) and the brain, kidney, lung, heart, liver, and spleen in 4-week-old mice, all tissues in which plexin-A1-transcripts are expressed (see FIGS. 2 and 3). These observations strongly suggest the existence of functional redundancy in the above tissues among the plexin family members during embryonic development. However, mutant mice had functional defects in the immune system as well as morphologic abnormalities in the skeletal tissues. Therefore, we investigated the biological functions of plexin-A1 further with a focus on the immune and skeletal tissues as described below.

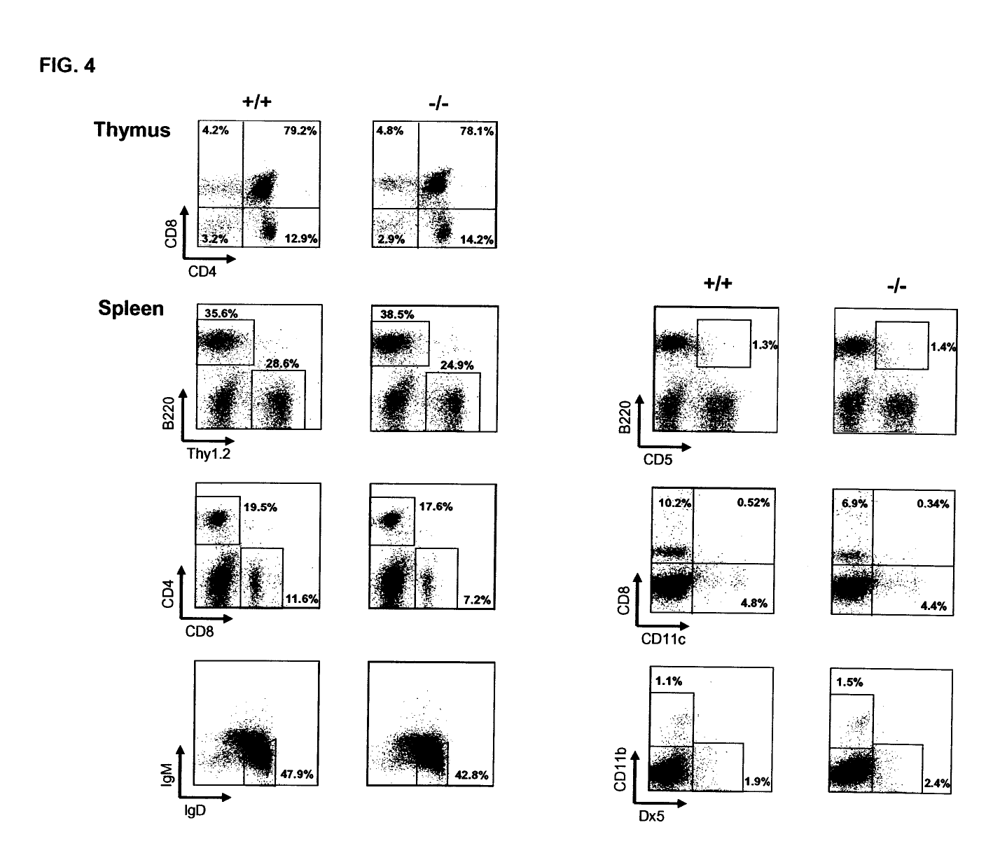

Lymphocyte development appeared to be normal in plexin-A1−/− mice. We did not observe any differences in the expression of cell surface phenotype markers, numbers and ratios of T-cells, B-cells, macrophages and Dendritic Cells (DCs) in the spleen and thymus between wild-type and plexin-A1−/− mice (see FIG. 4). Plexin-A1 is highly expressed in DCs¹⁷, and we examined the influence of plexin-A1-deficiency on DC functions. FITC-dextran-uptake by DCs and the appearance of fluorescent DCs in the draining lymph nodes after skin painting with FITC in plexin-A1−/− mice were comparable to those seen in wild-type littermates (FIGS. 16a and 16b). In addition, no significant differences were seen in the expression levels of co-stimulatory molecules, including CD40, CD80, CD86 and MHC class II, between wild-type and plexin-A1−/− DCs (FIG. 16c). However, plexin-A1−/− DCs poorly stimulated allogeneic T-cells compared to wild-type DCs (FIG. 17a). In contrast, when CD4+ T-cells from plexin-A1−/− mice or wild-type littermates were cultured with allogeneic wild-type DCs, no differences in cell proliferation were observed (FIG. 17b), suggesting an important role for DC-expressed plexin-A1 in stimulating allogeneic T-cells. In addition, the ability of plexin-A1−/− DCs to stimulate antigen-specific T cells in vitro was also impaired (FIG. 17c). These observations are consistent with the work of Wong et al. using RNAi-targeting plexin-A1 17; they showed that RNAi-mediated knock-down of plexin-A1 in DCs results in a substantial reduction in T-cell stimulation. Thus, the expression of plexin-A1 by DCs appears essential for normal T-cell stimulation. We next examined the generation of antigen (Ag)-specific T-cells in immunized plexin-A1−/− mice. CD4+ T-cells were prepared from the draining lymph nodes of immunized wild-type or plexin-A1−/− mice and Ag-specific T-cell responses were examined in vitro.

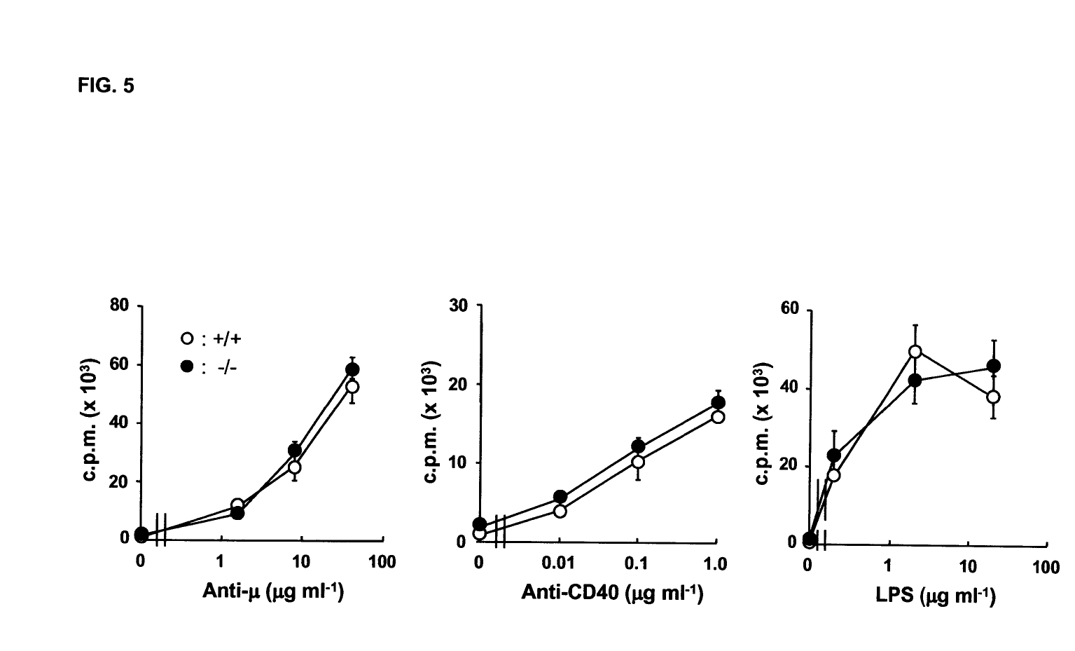

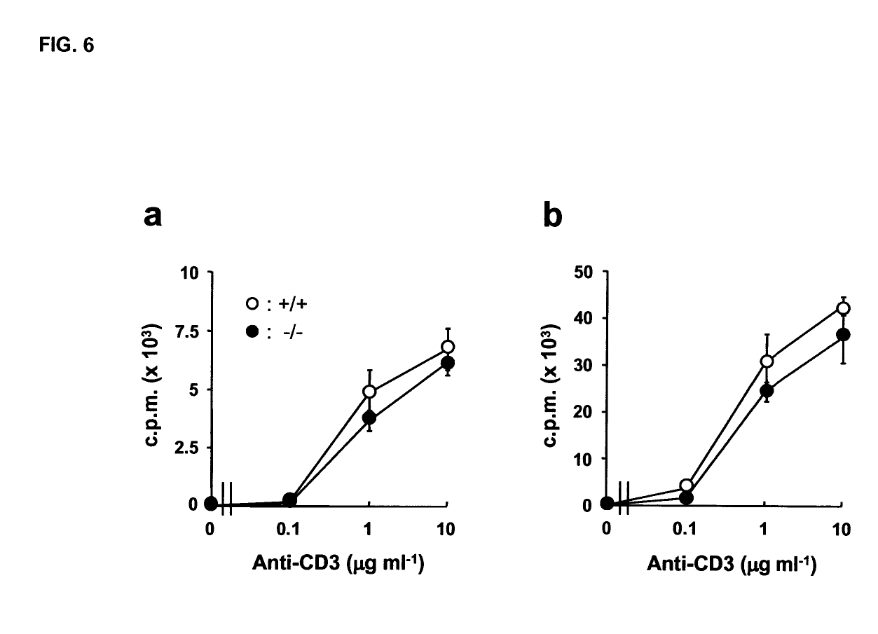

Proliferative responses and cytokine production by CD4+ T-cells were considerably reduced in plexin-A1−/− mice (FIG. 17d), demonstrating an important role for plexin-A1 in generating Ag-specific T-cells. In contrast, there were no differences in the in vitro responses of B- and T-cells to mitogenic stimulation between wild-type and plexin-A1−/− mice (see FIGS. 5 and 6), consistent with low levels of plexin-A1-expression in B- and T-cells 17 (Takegahara et al., unpublished data).

In the course of isolating bone marrow cells from plexin-A1−/− mice, we observed reduced cellularity (by 25±5%) in the long bones of plexin-A1−/− animals compared to wild-type littermates. In contrast, cell numbers and lymphocyte populations in the lymphoid organs were similar between mutant and control mice as described (see, FIG. 4). This role of plexin-A1 and it's function in bone homeostasis and bone resorption diseases was therefore investigated.

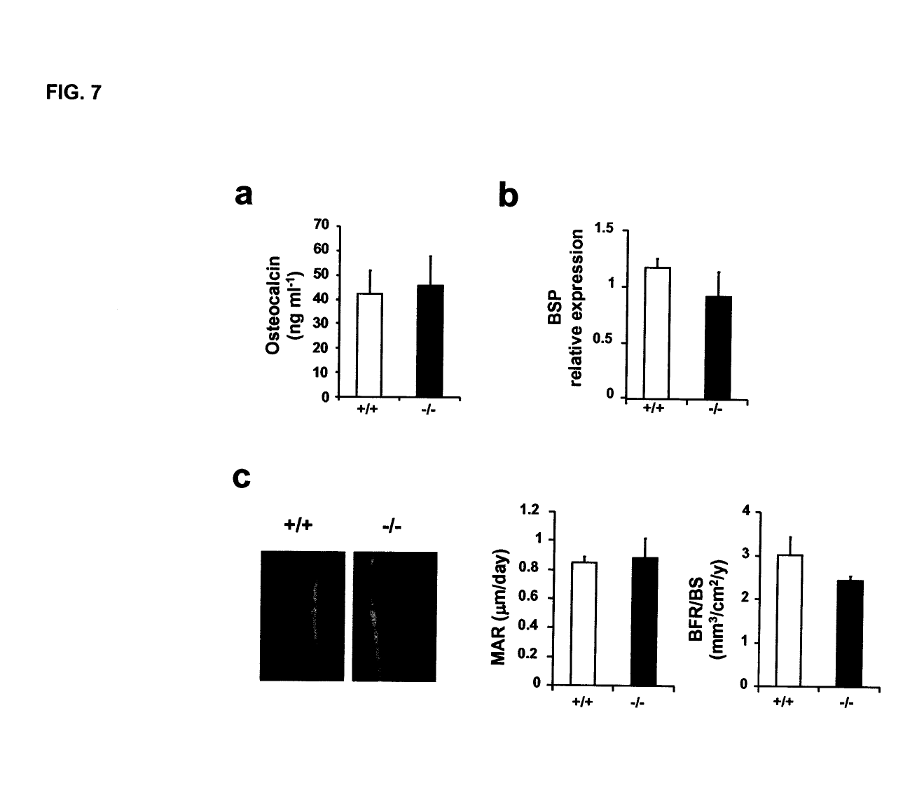

Three-dimensional microstructural analyses using high-resolution microcomputed tomography revealed that plexin-A1-deficiency unexpectedly resulted in increased bone mass (FIGS. 18a and 18b), which we confirmed using bone morphometric analyses (FIG. 18c). Sections of plexin-A1−/− long bones had increased trabecular mass compared to wild-type bones (FIG. 18d), indicating the development of osteopetrosis in plexin-A1−/− mice. The increased bone mass in plexin-A1−/− mice could be a consequence of increased osteoblast function, decreased osteoclast function, or both. To elucidate the cellular mechanism of the observed osteopetrosis in plexin-A1−/− mice, we examined the development and functions of osteoblasts and osteoclasts. Plexin-A1−/− osteoblasts promoted the formation of wild-type osteoclasts to the same extent as wild-type osteoblasts (FIG. 18e) and plexin-A1−/− osteoblasts isolated from calvarias had no obvious functional differences in the secretion of osteoclastogenic factors, including M-CSF and soluble receptor activator of NF-κB ligand (RANKL), and in vitro calcification (data not shown). There were no differences in the levels of osteoblast markers between wild-type and plexin-A1−/− mice (see FIG. 7). In addition, bone morphometric analysis showed normal ratios of osteoblast surface to bone surface (FIG. 18f). Calcein labelling also showed normal osteoblast activity in vivo (see FIG. 7).

Collectively, the loss of plexin-A1 had no apparent influence on osteoblast development and function. In contrast, histological bone morphometric analyses using TRAP-staining showed that plexin-A1−/− mice had considerably decreased osteoclast numbers and lower ratios of osteoclast surface to bone surface (FIG. 19a). In addition, plexin-A1−/− mice displayed a decrease in deoxypirydinoline (Dpyd) and collagen type I fragments (FIG. 19b), both of which are markers of osteoclast activity and bone resorption, indicating reduced in vivo bone turnover by ostoclasts. Consistent with this, the in vitro induction of osteoclasts^{18,19} was reduced in the absence of plexin-A1 (FIG. 19c). The number of TRAP-positive cells varied between individual mutant mice, however, and some mutant mice (˜40%) exhibited a normal number of TRAP-positive cells in vitro. The expression of all plexin-A members was seen in in vitro induced osteoclasts, while plexin-A1 was predominantly expressed in freshly isolated osteoclasts (FIG. 19d). It is possible that this variability might be due to the compensatory mechanisms involving other plexin-A family members.

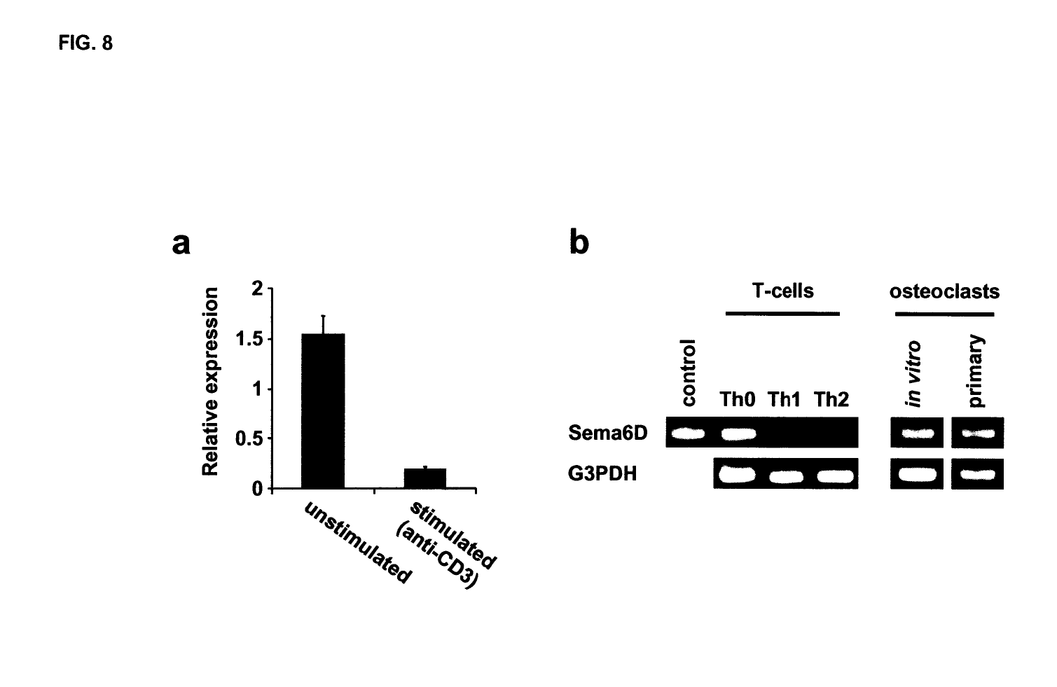

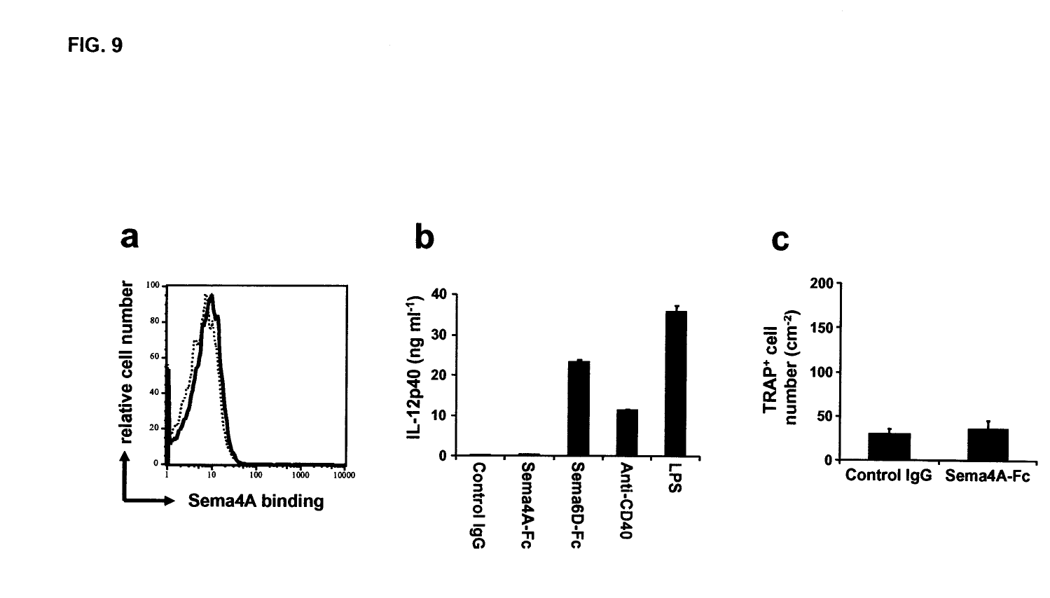

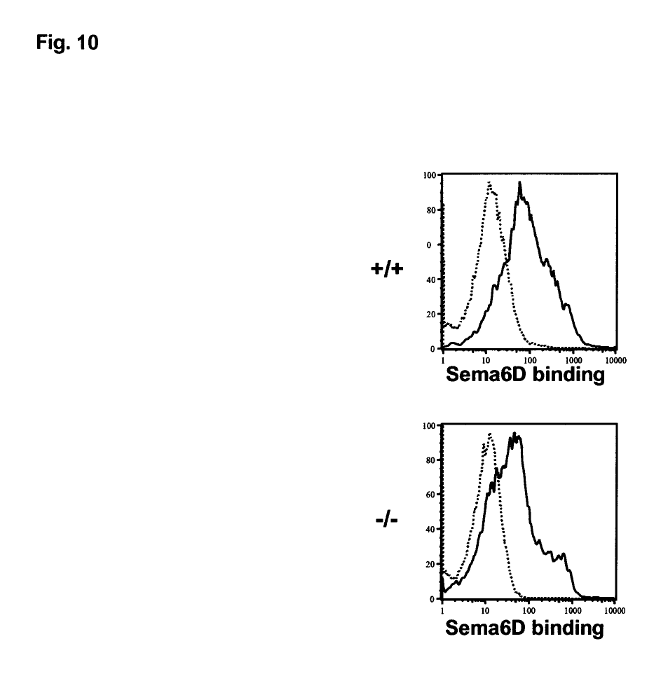

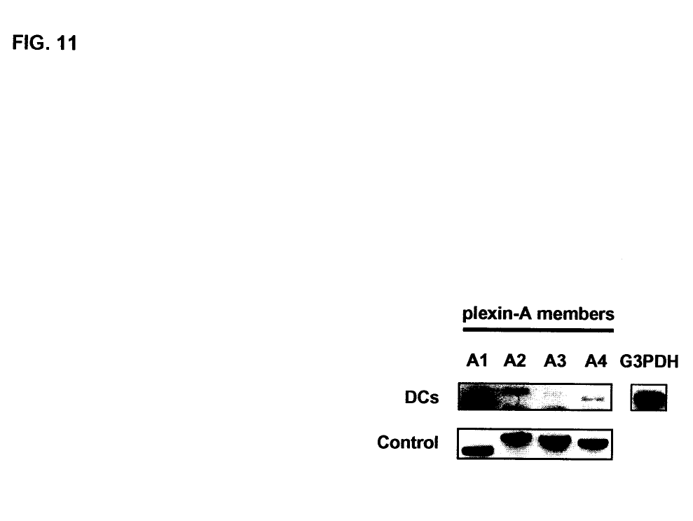

Plexin-A1 is clearly involved in the generation of immune responses and skeletal homeostasis, but the ligands responsible for these effects were unclear. In the nervous system, plexin-A1 associates with neuropilins, functioning as a signal transducing receptor component for class III semaphorins such as Sema3A^{2,15,20}. However, recombinant Sema3A neither promoted IL-12 production or co-stimulatory molecule expression on DCs, nor enhanced osteoclastogenesis in vitro (data not shown). Conversely, we previously identified plexin-A1 as a receptor for Sema6D during chick cardiac development¹⁶. In the immune system, Sema6D is highly expressed in T-cells (see FIG. 8), implying a role for Sema6D-plexin-A1 interactions in T-cell-DC cell-cell contacts. We thus examined the effects of soluble recombinant Sema6D on DC function. Incubation of DCs with recombinant soluble Sema6D 16 induced IL-12 production and the up-regulation of MHC class II-expression (FIG. 20a-20c), while such effects were not observed in control recombinant Sema4A proteins (see FIG. 9). In addition, Sema6D is expressed on osteoclasts (see FIG. 8), and soluble recombinant Sema6D promoted substantial osteoclast differentiation in vitro (FIG. 20d). Collectively, these results strongly suggest that plexin-A1 is a functional receptor for Sema6D in both the immune and skeletal tissues as well as during chick cardiac development. Consistent with this hypothesis, when plexin-A1−/− DCs were incubated with Sema6D, Sema6D binding, IL-12 production, and MHC class II up-regulation were all considerably reduced (FIG. 20a-20c). However, the residual Sema6D-binding was observed in plexin-A1−/− osteoclast precursors induced in vitro (see FIG. 10), thereby recombinant soluble Sema6D still promoted the in vitro induction of osteoclasts in the absence of plexin-A1 (data not shown). As previously described¹⁶, Sema6D can weakly bind other plexin-A subfamily members such as plexin-A4. The different responsiveness to Sema6D between DCs and osteoclasts in plexin-A1−/− mice is likely due to the expression of other plexin-A subfamily members in in vitro induced osteoclasts (FIG. 19d and see FIG. 11).

A common mechanism may underlie plexin-A1-function in the immune system and skeletal tissue. Alternatively, these functions may be unrelated. It is noteworthy that plexins utilize different co-receptors to exert a variety of biological effects^{2,16,21}. Indeed, plexin-A1 forms a receptor complex with receptor-type tyrosine kinases such as vascular endothelial growth factor receptor 2 (VEGFR2) or Off-track in a region-specific manner during chick cardiac morphogenesis. However, VEGFR2 and Off-track expression was not detected in DCs (data not shown). Therefore, plexin-A1 may associate with additional novel co-receptors to exert the functions described here.

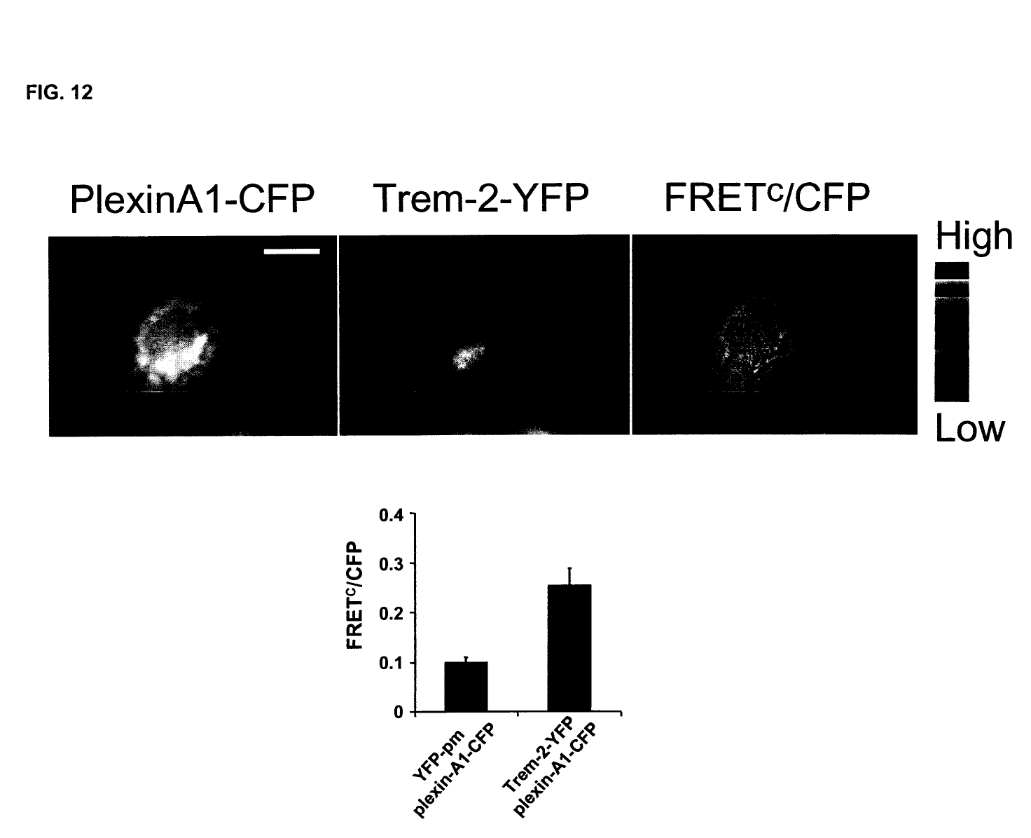

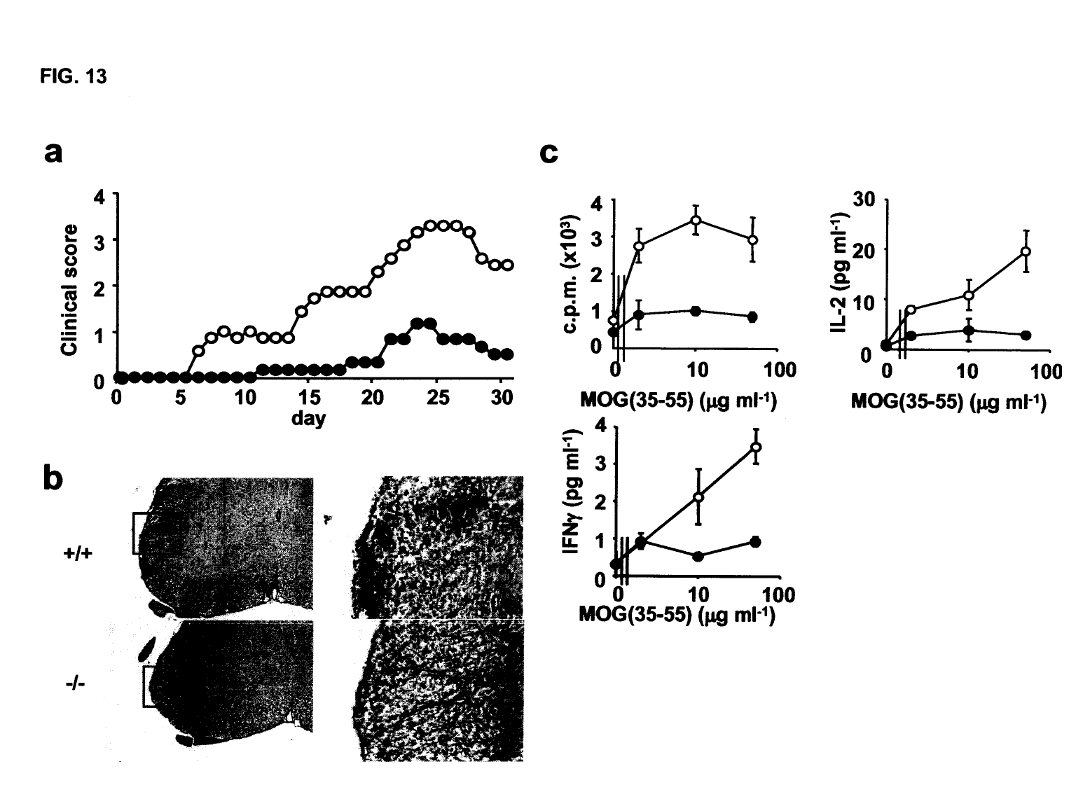

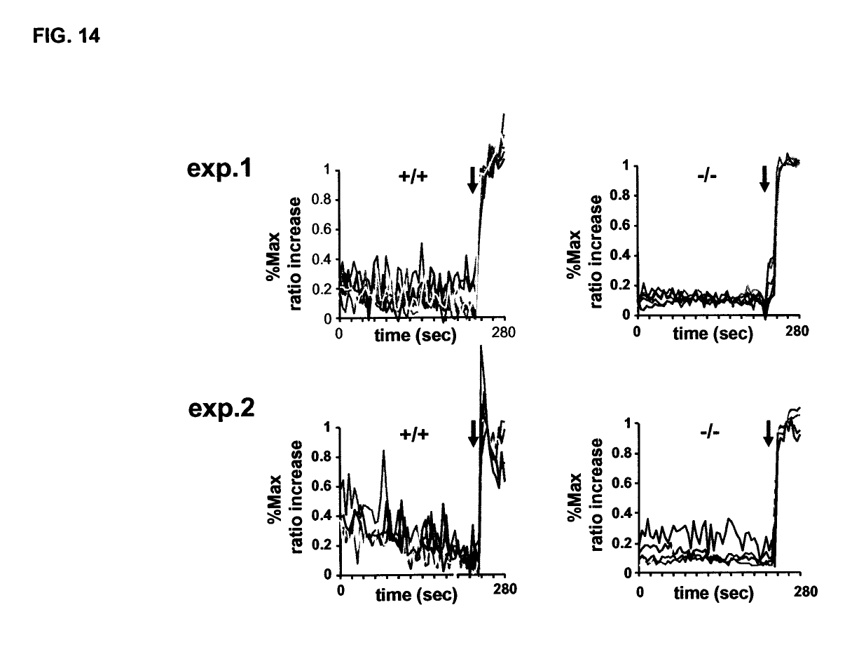

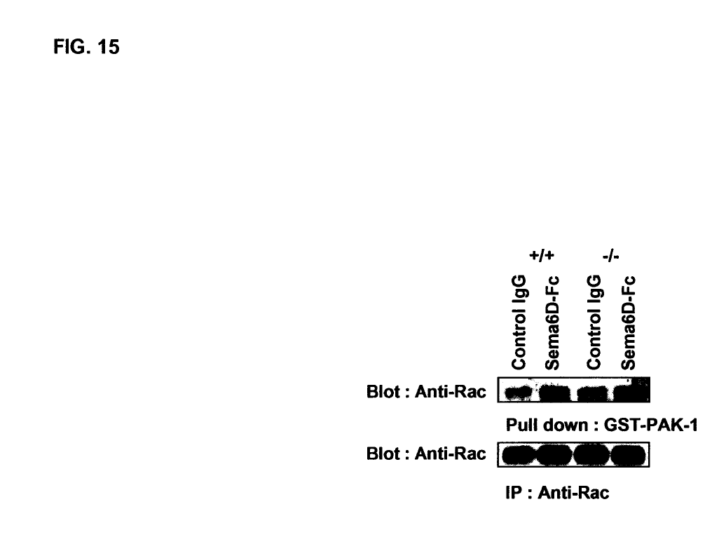

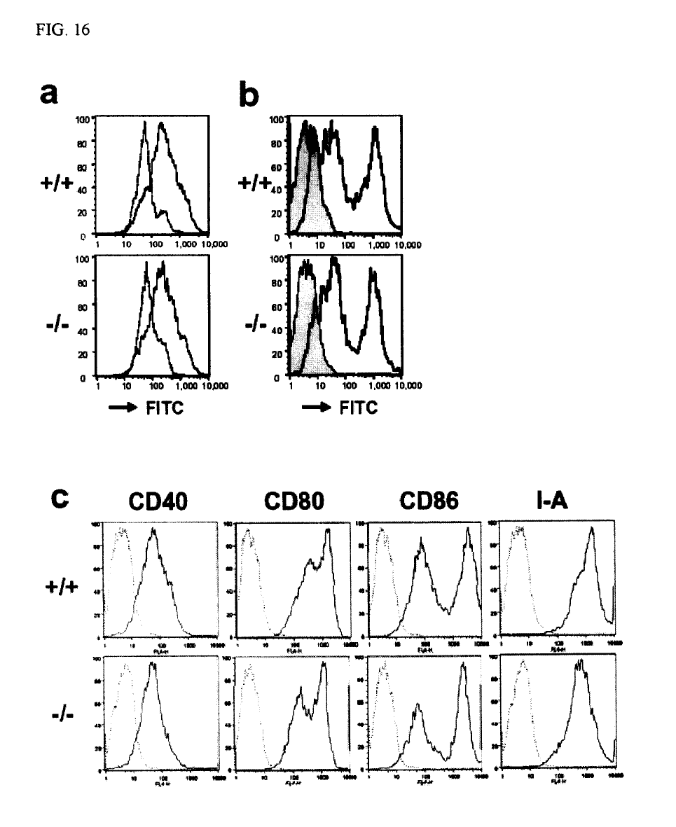

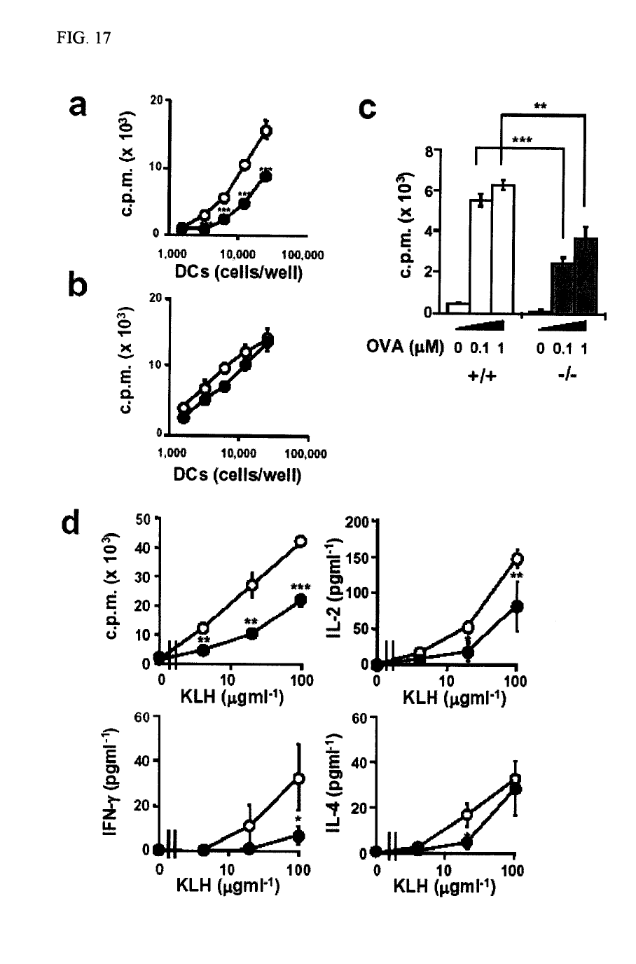

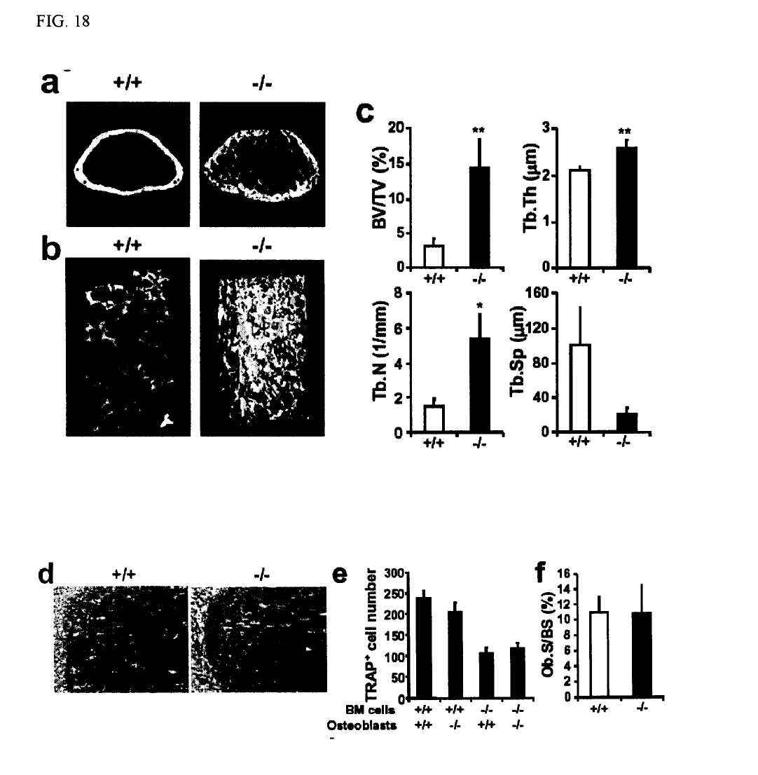

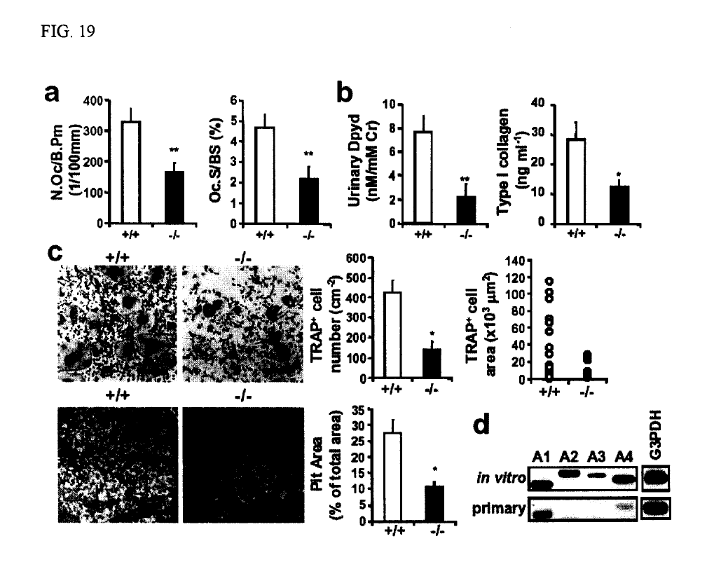

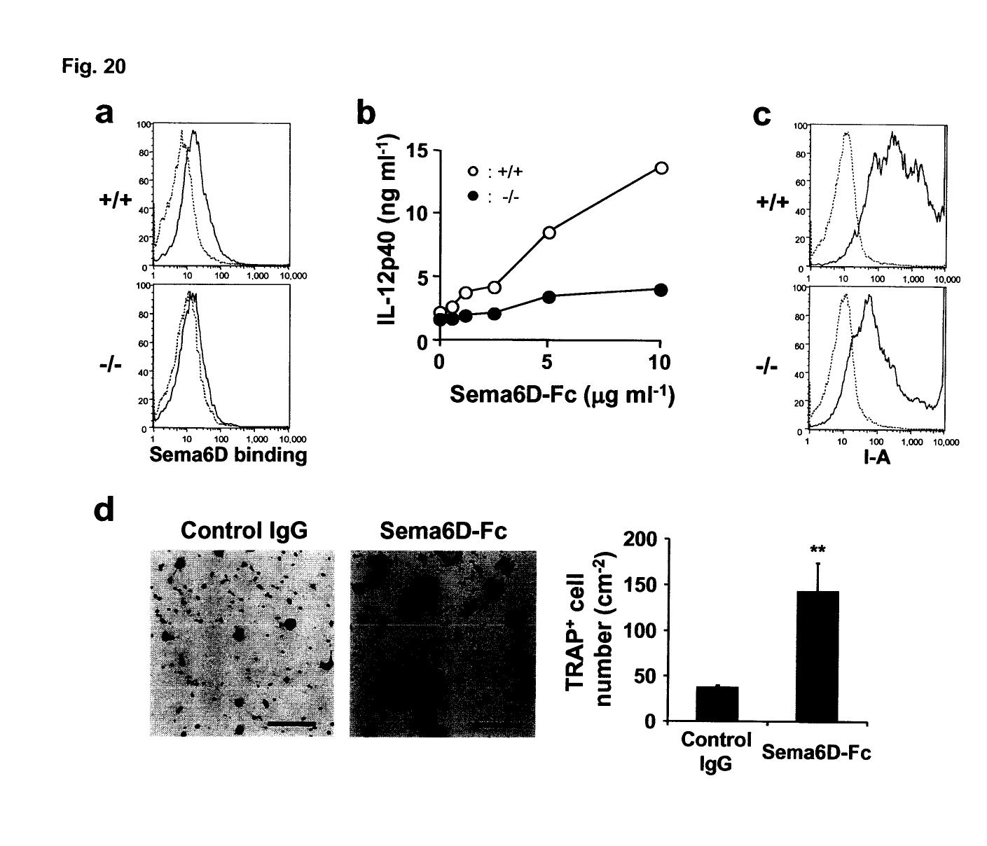

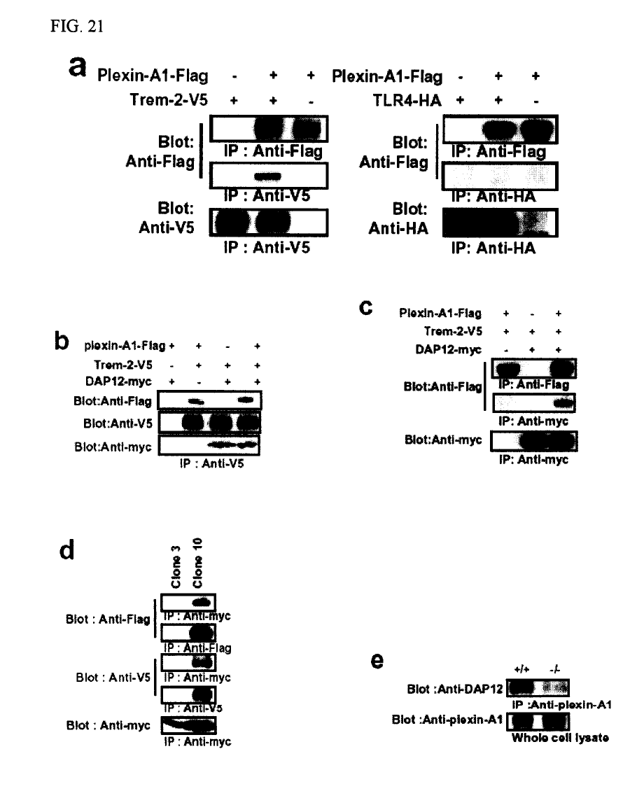

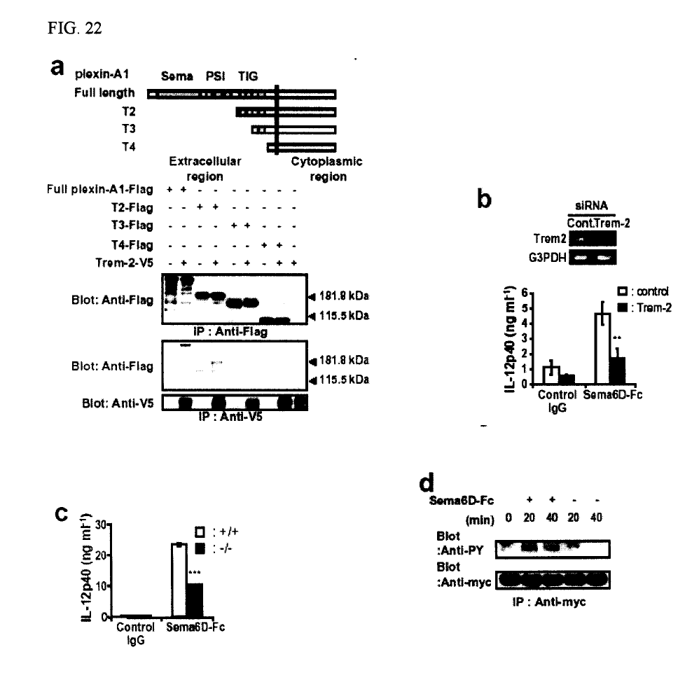

To better understand the mechanisms by which plexin-A1 affects both the immune system and bone homeostasis, we screened several candidate molecules with putative functions in both DCs and osteoclasts for association with plexin-A1. Using such an approach, we found that Trem-2 associated with plexin-A1 (FIG. 21a and FIG. 12). Therefore in a second generic embodiment, the invention also provides a method of treating an inflammatory, autoimmune or bone resorption disease, by administering to a patient a composition which inhibits plexin-A1-Trem-2 interaction.

Trem-2 forms a receptor complex with DAP12, an ITAM-bearing activating adaptor protein, via a positively-charged amino acid in its transmembrane domain^{22,23}. Interestingly, Trem-2 and DAP12 play critical roles not only in the development of immune responses but also in bone homeostasis by regulating osteoclast development^{24,25}. In COS7 cells transfected with plexin-A1, Trem-2 and DAP12, we observed the association of plexin-A1 with DAP12 in the presence of Trem-2 (FIGS. 21b and 21c), and this was also confirmed in cells stably expressing these proteins and DCs (FIGS. 21d and 21e). To determine the structural requirements for the association of plexin-A1 and Trem-2, we co-transfected constructs encoding Trem-2 with a series of N-terminal truncation mutants of plexin-A1. As shown in FIG. 4f, the association of plexin-A1 with Trem-2 was still detected even in the absence of the Sema and plexin/semaphorin/integrin (PSI) domains of plexin-A1, although the association was considerably reduced. The association, however, was completely abolished by deletion of the plexin-A1 TIG domain. It thus appears that the plexin-A1-TIG domain is minimally required for the interaction of plexin-A1 with Trem-2.

In yet another embodiment of the invention there is provided a method of treating an inflammatory, autoimmune or bone resorption disease, by administering to a patient a composition which inhibits DAP12-Trem-2 interaction.

In order to determine the role of Trem-2 and DAP12 in semaphorin-mediated signals, we performed a ‘loss of function’ experiment. RNAi against Trem-2considerably reduced the stimulatory activities of Sema6D on DCs (FIG. 22b). Similarly, DAP12−/− DCs exhibited considerably reduced responses to Sema6D (FIG. 22c). When RAW264.7 cells expressing plexin-A1, Trem-2 and DAP12 were stimulated with recombinant soluble Sema6D protein, tyrosine phosphorylation of DAP12 was observed (FIG. 22d). Collectively, these results strongly suggest that DAP12 and Trem-2 are functional receptor components for Sema6D.

Plexin-A1 is expressed in a broad range of tissues from embryos to adults (see FIG. 3), and a role for plexin-A1 in axon guidance and cardiac morphogenesis during development has been suggested^{15,16}. However, our present study has revealed that the developmental or functional defects of plexin-A1−/− mice are primarily restricted to the immune and skeletal tissues. Our failure to detect defects in the nervous and cardiovascular systems may be due to compensatory mechanisms by other plexin family members. In addition, there is a possibility that the mutant mice may have subtle defects that were overlooked in our gross macroscopic and histological analyses. More detailed examination of the mutant mice is required to answer these questions. Either Ag-uptake or expression levels of costimulatory molecules on plexin-A1−/− DCs were comparable to those on wild-type DCs (FIG. 16a-16c), indicating that plexin-A1 is not involved in the development of DCs. However, the allogeneic and Ag-specific T-cell stimulatory activities of DCs were impaired in plexin-A1−/− mice (FIGS. 17a and 17c), suggesting that the deficiency of plexin-A1 on DCs is primarily responsible for the impaired activity of DCs to stimulate T-cells. In this context, the reduced stimulatory activities of plexin-A1−/− DCs could explain the defective T-cell priming in plexin-A1−/− mice. Of note, Sema6D is abundantly expressed on T-cells but is down-regulated during T helper cell (Th) differentiation (see FIG. 8), suggesting the involvement of Sema6D-plexin-A1 interactions in relatively early phases of immune responses through T-cell-DC contacts. However, at the moment, we can not exclude the possible involvement of Sema6D-plexin-A1 interactions in effector phases of immune responses.

The expression of Sema6D was detected not only in in vitro induced osteoclasts but also in freshly isolated osteoclasts (see FIG. 8). However, it is noteworthy that, although Sema6D is a transmembrane-type semaphorin, it has been demonstrated that a soluble form of Sema6D is cleaved from the cell surface¹⁶. Thus, Sema6D appears to act in osteoclastogenesis in an autocrine manner. In this regard, it is possible that Sema6D can function in both autocrine and paracrine manners during osteoclastogenesis. However, it remains unclear if the effects of Sema6D are different depending on the autocrine versus paracrine stimulation. Also, further studies will be required to know whether the biological activity of the transmembrane-type Smea6D is functionally different from that of soluble Sema6D.

One embodiment of the present invention relates to a method to identify a compound that controls interaction of plexin-A1 with DAP12 activity in a cell, comprising: (1) contacting a cell with a putative regulatory compound, wherein the cell includes a plexin-A1 protein and a DAP12 protein; and (2) assessing the ability of the putative regulatory compound to inhibit the interaction of plexin-A1 with DAP12. The assessment step preferably involves either i) determining the cytokine production as described herein-below, ii) in vitro osteoclastogenesis performed as described previously^{24,25} and methods known in the art.

Another embodiment of the present invention relates to a method to identify a compound that controls interaction of plexin-A1 with Trem-2 activity in a cell, comprising: (1) contacting a cell with a putative regulatory compound, wherein the cell includes a plexin-A1 protein and a Trem-2 protein; and (2) assessing the ability of the putative regulatory compound to inhibit the interaction of plexin-A1 with Trem-2. The assessment step preferably involves either i) determining the cytokine production as described herein-below, ii) in vitro osteoclastogenesis performed as described previously^{24,25} and methods known in the art.

Yet another embodiment of the present invention relates to a method to identify a compound that controls interaction of DAP12 with Trem-2 activity in a cell, comprising: (1) contacting a cell with a putative regulatory compound, wherein the cell includes a DAP12 protein and a Trem-2 protein; and (2) assessing the ability of the putative regulatory compound to inhibit the interaction of DAP12 with Trem-2. The assessment step preferably involves either i) determining the cytokine production as described herein-below, ii) in vitro osteoclastogenesis performed as described previously^{24,25} and methods known in the art.

The term “regulate” refers to controlling the activity of a molecule and/or biological function, such as enhancing or diminishing such activity or function.

The term “patient” includes both human and non-human mammals.

The terms “treating” or “treatment” mean the treatment of a disease-state in a patient, and include:
(i) preventing the disease-state from occurring in a patient, in particular, when such patient is genetically or otherwise predisposed to the disease-state but has not yet been diagnosed as having it;
(ii) inhibiting or ameliorating the disease-state in a patient, i.e., arresting or slowing its development; or
(iii) relieving the disease-state in a patient, i.e., causing regression or cure of the disease-state.

Yet another embodiment of the present invention relates to an antibody or antibody binding site which binds plexin-A1, Trem-2 or DAP12 or fragments thereof. Embodiments of the present invention further include polyclonal and monoclonal antibodies. Preferred embodiments of the present invention include a monoclonal antibody such an anti-plexin-A1 monoclonal antibody. The above antibody or antibody binding site which binds plexin-A1, Trem-2 or DAP12 inhibits binding of plexin-A1 to DAP12 or Trem-2, or Trem-2 binding to DAP12.

Yet another embodiment of the present invention relates to a biotherapeutic comprising plexin-A1 protein, Trem-2 protein or DAP12 protein or fragments thereof, wherein the biotherapeutic is useful for treating an inflammatory, autoimmune or bone resorption disease.

The term “composition” as referred to herein include a putative compound, or a substantially pure protein selected from plexin-A1, Trem-2 or DAP12 or fragments thereof, an antibody or antibody binding site which binds plexin-A1, Trem-2 or DAP12 or fragments thereof, to an expression vector encoding plexin-A1, Trem-2 or DAP12 or fragments thereof, a fusion protein comprising plexin-A1, Trem-2 or DAP12 or fragments thereof. In the antibody binding site embodiments, the antibody binding site may be: specifically immunoreactive with a mature protein selected from the group consisting of the plexin-A1, Trem-2 or DAP12; raised against a purified or recombinantly produced human or mouse plexin-A1, Trem-2 or DAP12; in a monoclonal antibody, Fab, or F(ab)2; immunoreactive with denatured antigen; or in a labeled antibody. In certain embodiments; the antibody binding site is detected in a biological sample by a method of: contacting a binding agent having an affinity for plexin-A1, Trem-2 or DAP12 with the biological sample; incubating the binding agent with the biological sample to form a binding agent: plexin-A1, Trem-2 or DAP12 protein complex; and detecting the complex. In a preferred embodiment, the biological sample is human, and the binding agent is an antibody.

Putative compounds as referred to herein include, for example, compounds that are products of rational drug design, natural products and compounds having partially defined signal transduction regulatory properties. A putative compound can be a protein-based compound, a carbohydrate-based compound, a lipid-based compound, a nucleic acid-based compound, a natural organic compound, a synthetically derived organic compound, an anti-idiotypic antibody and/or catalytic antibody, or fragments thereof. A putative regulatory compound can be obtained, for example, from libraries of natural or synthetic compounds, in particular from chemical or combinatorial libraries (i.e., libraries of compounds that differ in sequence or size but that have the same building blocks; see for example, U.S. Pat. Nos. 5,010,175 and 5,266,684 of Rutter and Santi, which are incorporated herein by reference in their entirety) or by rational drug design.

In a rational drug design procedure, the three-dimensional structure of a compound, such as a signal transduction molecule can be analyzed by, for example, nuclear magnetic resonance (NMR) or x-ray crystallography. This three-dimensional structure can then be used to predict structures of potential compounds, such as putative regulatory compounds by, for example, computer modelling. The predicted compound structure can then be produced by, for example, chemical synthesis, recombinant DNA technology, or by isolating a mimetope from a natural source (e.g., plants, animals, bacteria and fungi). Potential regulatory compounds can also be identified using SELEX technology as described in, for example, PCT Publication Nos. WO 91/19813; WO 92/02536 and WO 93/03172 (which are incorporated herein by reference in their entirety).

In particular, a naturally-occurring intracellular signal transduction molecule can be modified based on an analysis of its structure and function to form a suitable regulatory compound. For example, a compound capable of regulating the plexin-A1-TIG domain can comprise a compound having similar structure to the amino acid residues in this domain. Such a compound can comprise a peptide, a polypeptide or a small organic molecule.

Putative regulatory compounds can also include molecules designed to interfere with plexin-A1. For example, mutants of plexin-A1 can be created that interfere with the coupling of the protein with Trem-2 and or DAP12. Putative regulatory compounds can include agonists and antagonists of plexin-A1, Trem-2 or DAP12. Such agonists and antagonists can be selected based on the structure of a naturally-occurring ligand to these proteins.

The technology for producing monoclonal antibodies is well known. In general, an immortal cell line (typically myeloma cells) is fused to lymphocytes (typically splenocytes) from a mammal immunized with whole cells expressing a given antigen, e.g., plexin-A1, and the culture supernatants of the resulting hybridoma cells are screened for antibodies against the antigen. See, generally, Kohler et at., 1975, Nature 265: 295-497, “Continuous Cultures of Fused Cells Secreting Antibody of Predefined Specificity”.

Immunization may be accomplished using standard procedures. The unit dose and immunization regimen depend on the species of mammal immunized, its immune status, the body weight of the mammal, etc. Typically, the immunized mammals are bled and the serum from each blood sample is assayed for particular antibodies using appropriate screening assays. For example, anti-integrin antibodies may be identified by immunoprecipitation of 125I-labeled cell lysates from integrin-expressing cells. Antibodies, including for example, anti-plexin-A1 antibodies, may also be identified by flow cytometry, e.g., by measuring fluorescent staining of antibody-expressing cells incubated with an antibody believed to recognize plexin-A1 molecules. The lymphocytes used in the production of hybridoma cells typically are isolated from immunized mammals whose sera have already tested positive for the presence of anti-plexin-A1 antibodies using such screening assays.

Typically, the immortal cell line (e.g., a myeloma cell line) is derived from the same mammalian species as the lymphocytes. Preferred immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion are then selected using HAT medium, which kills unfused and unproductively fused myeloma cells (unfused splenocytes die after several days because they are not transformed). Hybridomas producing a desired antibody are detected by screening the hybridoma culture supernatants. For example, hybridomas prepared to produce anti-plexin-A1 antibodies may be screened by testing the hybridoma culture supernatant for secreted antibodies having the ability to bind to a recombinant plexin-A1-expressing cell line.

To produce antibody homologs which are within the scope of the invention, including for example, anti-plexin-A1 antibody homologs, that are intact immunoglobulins, hybridoma cells that tested positive in such screening assays were cultured in a nutrient medium under conditions and for a time sufficient to allow the hybridoma cells to secrete the monoclonal antibodies into the culture medium. Tissue culture techniques and culture media suitable for hybridoma cells are well known. The conditioned hybridoma culture supernatant may be collected and the anti-plexin-A1 antibodies optionally further purified by well-known methods.

Alternatively, the desired antibody may be produced by injecting the hybridoma cells into the peritoneal cavity of an unimmunized mouse. The hybridoma cells proliferate in the peritoneal cavity, secreting the antibody which accumulates as ascites fluid. The antibody may be harvested by withdrawing the ascites fluid from the peritoneal cavity with a syringe.

Fully human monoclonal antibody homologs against, for example plexin-A1, are another preferred binding agent which may block antigens in the method of the invention. In their intact form these may be prepared using in vitro-primed human splenocytes, as described by Boerner et al., 1991, J. Immunol. 147:86-95, “Production of Antigen-specific Human Monoclonal Antibodies from In Vitro-Primed Human Splenocytes”.

Alternatively, they may be prepared by repertoire cloning as described by Persson et al., 1991, Proc. Nat. Acad. Sci. USA 88: 2432-2436, “Generation of diverse high-affinity human monoclonal antibodies by repertoire cloning” and Huang and Stollar, 1991, J. Immunol. Methods 141: 227-236, “Construction of representative immunoglobulin variable region CDNA libraries from human peripheral blood lymphocytes without in vitro stimulation”. U.S. Pat. No. 5,798,230 (Aug. 25, 1998, “Process for the preparation of human monoclonal antibodies and their use”) describes preparation of human monoclonal antibodies from human B cells. According to this process, human antibody-producing B cells are immortalized by infection with an Epstein-Barr virus, or a derivative thereof, that expresses Epstein-Barr virus nuclear antigen 2 (EBNA2). EBNA2 function, which is required for immortalization, is subsequently shut off, which results in an increase in antibody production.

In yet another method for producing fully human antibodies, U.S. Pat. No. 5,789,650 (Aug. 4, 1998, “Transgenic non-human animals for producing heterologous antibodies”) describes transgenic non-human animals capable of producing heterologous antibodies and transgenic non-human animals having inactivated endogenous immunoglobulin genes. Endogenous immunoglobulin genes are suppressed by antisense polynucleotides and/or by antiserum directed against endogenous immunoglobulins. Heterologous antibodies are encoded by immunoglobulin genes not normally found in the genome of that species of non-human animal. One or more transgenes containing sequences of unrearranged heterologous human immunoglobulin heavy chains are introduced into a non-human animal thereby forming a transgenic animal capable of functionally rearranging transgenic immunoglobulin sequences and producing a repertoire of antibodies of various isotypes encoded by human immunoglobulin genes. Such heterologous human antibodies are produced in B-cells which are thereafter immortalized, e.g., by fusing with an immortalizing cell line such as a myeloma or by manipulating such B-cells by other techniques to perpetuate a cell line capable of producing a monoclonal heterologous, fully human antibody homolog.

The conditions under which the cell of the present invention is contacted with a putative regulatory compound, such as by mixing, are conditions in which the cell can exhibit plexin-A1, Trem-2 or DAP12 activity if essentially no other regulatory compounds are present that would interfere with such activity. Achieving such conditions is within the skill in the art, and includes an effective medium in which the cell can be cultured such that the cell can exhibit plexin-A1, Trem-2 or DAP12 activity. For example, for a mammalian cell, effective media are typically aqueous media comprising RPMI 1640 medium containing 10% fetal calf serum.

Cells of the present invention can be cultured in a variety of containers including, but not limited to, tissue culture flasks, test tubes, microtiter dishes, and petri plates. Culturing is carried out at a temperature, pH and carbon dioxide content appropriate for the cell. Such culturing conditions are also within the skill in the art. For example, for Ramos cells, culturing can be carried out at 37.degree. C., in a 5% CO.sub.2 environment.

Acceptable protocols to contact a cell with a putative regulatory compound in an effective manner include the number of cells per container contacted, the concentration of putative regulatory compound(s) administered to a cell, the incubation time of the putative regulatory compound with the cell, the concentration of ligand and/or intracellular initiator molecules administered to a cell, and the incubation time of the ligand and/or intracellular initiator molecule with the cell. Determination of such protocols can be accomplished by those skilled in the art based on variables such as the size of the container, the volume of liquid in the container, the type of cell being tested and the chemical composition of the putative regulatory compound (i.e., size, charge etc.) being tested.

In one embodiment of the method of the present invention, a suitable number of cells are added to a 96-well tissue culture dish in culture medium. A preferred number of cells includes a number of cells that enables one to detect a change in plexin-A1, Trem-2 or DAP12 activity using a detection method of the present invention (described in detail below). A more preferred number of cells includes between about 1 and 1.times.10.sup.6 cells per well of a 96-well tissue culture dish. Following addition of the cells to the tissue culture dish, the cells can be preincubated at 37.degree. C., 5% C.sub.2O for between about 0 to about 24 hours.

A suitable amount of putative regulatory compound(s) suspended in culture medium is added to the cells that is sufficient to regulate the activity of a plexin-A1, Trem-2 or DAP12 protein in a cell such that the regulation is detectable using a detection method of the present invention. A preferred amount of putative regulatory compound(s) comprises between about 1 nM to about 10 mM of putative regulatory compound(s) per well of a 96-well plate. The cells are allowed to incubate for a suitable length of time to allow the putative regulatory compound to enter a cell and interact with plexin-A1, Trem-2 or DAP12 protein. A preferred incubation time is between about 1 minute to about 48 hours.

In another embodiment of the method of the present invention, cells suitable for use in the present invention are stimulated with a stimulatory molecules capable of binding to plexin-A1, Trem-2 or DAP12 protein of the present invention to initiate a signal transduction pathway and create a cellular response. Preferably, cells are stimulated with a stimulatory molecule following contact of a putative regulatory compound with a cell. Suitable stimulatory molecules can include, for example, antibodies that bind specifically to plexin-A1, Trem-2 or DAP12 protein. A suitable amount of stimulatory molecule to add to a cell depends upon factors such as the type of ligand used (e.g., monomeric or multimeric; permeability, etc.) and the abundance of plexin-A1, Trem-2 or DAP12 protein. Preferably, between about 1.0 nM and about 1 mM of ligand is added to a cell.

The method of the present invention include determining if a composition is capable of regulating plexin-A1, Trem-2 or DAP12 protein activation. Such methods include assays described in detail in the Examples section. The method of the present invention can further include the step of performing a toxicity test to determine the toxicity of the composition.

Another aspect of the present invention includes a kit to identify compositions capable of regulating plexin-A1, Trem-2 or DAP12 protein activity in a cell. Such a kit includes: (1) a cell comprising plexin-A1, Trem-2 or DAP12 protein; and (2) a means for detecting regulation of either the plexin-A1, Trem-2 or DAP12 protein. Such a means for detecting the regulation of plexin-A1, Trem-2 or DAP12 protein include methods and reagents known to those of skill in the art, for example, plexin-A1 protein activity can be detected using, for example, activation assays described herein-below. Means for detecting the regulation of plexin-A1, Trem-2 or DAP12 protein also include methods and reagents known to those of skill in the art. Suitable cells for use with a kit of the present invention include cells described in detail herein. A preferred cell for use with a kit includes a human cell.

### Methods of Therapeutic Use

It has been found for the first time by the present inventors that plexin-A1 can associate with DAP12, in both the development of normal immune responses and bone homeostasis.

ITAM-mediated signaling through DAP12 has been previously shown to be an important co-stimulatory signal not only for the proper development of immune responses but also for osteoclast differentiation²⁴⁻²⁶. As reported in DAP12−/− mice²⁶, plexin-A1−/− mice displayed impaired generation of Ag-specific T-cells, in which they were resistant to the development of experimental autoimmune encephalomyelitis (EAE) (see FIG. 13). Also in the skeletal tissues, the defects in the DAP12 gene as well as Trem-2 gene are known to result in impaired differentiation of osteoclasts^{24,25,27,28}. Thus, the association of plexin-A1 with DAP12 likely provides co-stimulatory signals to both DCs and osteoclasts. In support of this, calcium signaling was affected in plexin-A1−/− cells (see FIG. 12,14) as it is the case for DAP12−/− cells²⁵. The invention therefore provides a method of treating an inflammatory, autoimmune or bone resorption disease, by administering to a patient a composition which inhibits plexin-A1-DAP12 interaction.

The present inventors have also shown that Trem-2 acts as a bridge for the plexin-A1-DAP12 association. The invention therefore also provides a method of treating an inflammatory, autoimmune or bone resorption disease, by administering to a patient a composition which inhibits plexin-A1-Trem-2 interaction.

The present inventors have also demonstrated that that DAP12 and Trem-2 are functional receptor components for Sema6D. The invention therefore also provides a method of treating an inflammatory, autoimmune or bone resorption disease, by administering to a patient a composition which inhibits DAP12-Trem-2 interaction.

A composition which would block the interaction of plexin-A1 with DAP12, plexin-A1-Trem-2, or DAP12-Trem-2 would block inflammatory cytokine production from cells. The inhibition of cytokine production is an attractive means for preventing and treating a variety of cytokine mediated diseases or conditions associated with excess cytokine production, e.g., diseases and pathological conditions involving inflammation, autoimmune responses or bone resorption. Thus, the compositions are useful for the treatment of diseases and conditions including the following:

osteoarthritis, atherosclerosis, contact dermatitis, bone resorption diseases including osteoporosis, reperfusion injury, asthma, multiple sclerosis, Guillain-Barre syndrome, Crohn's disease, ulcerative colitis, psoriasis, graft versus host disease, systemic lupus erythematosus and insulin-dependent diabetes mellitus, rheumatoid arthritis, toxic shock syndrome, Alzheimer's disease, diabetes, inflammatory bowel diseases, acute and chronic pain as well as symptoms of inflammation and cardiovascular disease, stroke, myocardial infarction, alone or following thrombolytic therapy, thermal injury, adult respiratory distress syndrome (ARDS), multiple organ injury secondary to trauma, acute glomerulonephritis, dermatoses with acute inflammatory components, acute purulent meningitis or other central nervous system disorders, syndromes associated with hemodialysis, leukopherisis, granulocyte transfusion associated syndromes, and necrotizing entrerocolitis, complications including restenosis following percutaneous transluminal coronary angioplasty, traumatic arthritis, sepsis, chronic obstructive pulmonary disease and congestive heart failure. Said composition may also be useful for anticoagulant or fibrinolytic therapy (and the diseases or conditions related to such therapy).

Anti-cytokine activity can be demonstrated by using methods known in the art. See for example Branger et al., (2002) J Immunol. 168: 4070-4077, and the 46 references cited therein, each incorporated herein by reference in their entirety.

A composition according to the invention will also be useful for treating oncological diseases. These diseases include but are not limited to solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma and mesothelioma.

Examples of brain cancers include, but are not limited to brain stem, optic and hypophtalmic glioma, cerebella and cerebral astrocytoma, medulloblastoma, ependymoma, as well as pituitary, neuroectodermal and pineal tumor.

Examples of peripheral nervous system tumors include, but are not limited to neuroblastoma, ganglioneuroblastoma, and peripheral nerve sheath tumors.

Examples of tumors of the endocrine and exocrine system include, but are not limited to thyroid carcinoma, adrenocortical carcinoma, pheochromocytoma, and carcinoid tumors.

Tumors of the male reproductive organs include, but are not limited to prostate and testicular cancer.

Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallblader, gastric, pancreatic, rectal, small-intestine, and salivary gland cancers.

Tumors of the urinary tract include, but are not limited to bladder, penile, kidney, renal pelvis, ureter, and urethral cancers.

Eye cancers include, but are not limited to intraocular melanoma and retinoblastoma.

Examples of liver cancers include, but are not limited to hepatocellular carcinoma (liver cell carcinomas with or without fibrolamellar variant), hepatoblastoma, cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal/hypopharyngeal/nasopharyngeal/oropharyngeal cancer, and lip and oral cavity cancer.

Lymphomas include, but are not limited to AIDS-related lymphoma, non-Hodgkin's lymphoma, Hodgkins lymphoma, cutaneous T-cell lymphoma, and lymphoma of the central nervous system.

Sarcomas include, but are not limited to sarcoma of the soft tissue, osteosarcoma, Ewings sarcoma, malignant fibrous histiocytoma, lymphosarcoma, angiosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia.

Plasma cell dyscrasias include, but are not limited to multiple myeloma, and Waldenstrom's macroglobulinemia.

These disorders have been well characterized in man, but also exist with a similar etiology in other mammals, and can be treated by pharmaceutical compositions of the present invention.

For therapeutic use, the compositions may be administered in any conventional dosage form in any conventional manner. Routes of administration include, but are not limited to, intravenously, intramuscularly, subcutaneously, intrasynovially, by infusion, sublingually, transdermally, orally, topically or by inhalation. The preferred modes of administration are oral and intravenous.

The compositions may be administered alone or in combination with adjuvants that enhance stability of the inhibitors, facilitate administration of pharmaceutic compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies. The above described compositions may be physically combined with the conventional therapeutics or other adjuvants into a single pharmaceutical composition. Advantageously, the compositions may then be administered together in a single dosage form. In some embodiments, the pharmaceutical compositions comprising such combinations of compositions contain at least about 5%, but more preferably at least about 20%, of a composition (w/w) or a combination thereof. The optimum percentage (w/w) of a composition of the invention may vary and is within the purview of those skilled in the art. Alternatively, the compositions may be administered separately (either serially or in parallel). Separate dosing allows for greater flexibility in the dosing regime.

As mentioned above, dosage forms of the compositions described herein include pharmaceutically acceptable carriers and adjuvants known to those of ordinary skill in the art. These carriers and adjuvants include, for example, ion exchangers, alumina, aluminum stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 1-1000 mg/dose for a 70 kg patient. Although one dose per day may be sufficient, up to 5 doses per day may be given. For oral doses, up to 2000 mg/day may be required. As the skilled artisan will appreciate, lower or higher doses may be required depending on particular factors. For instance, specific dosage and treatment regimens will depend on factors such as the patient's general health profile, the severity and course of the patient's disorder or disposition thereto, and the judgment of the treating physician.

Experimental Methods

Mice

To construct the plexin-A1 targeting vector, a 3-kb fragment containing the second exon with the initiation codon and third exon with the coding sequence of the sema-domain was replaced with the neo resistance cassette, and the Herpes simplex virus thymidine kinase (HSV-tk) gene was inserted for selection against random integration. The linearized targeting plasmid DNA was transfected into ES cells by electroporation. After double selection with G418 and gancyclovir, 96 resistant clones were screened for homologous recombination of the plexin-A1 targeted allele by PCR and Southern blot analysis as described below. Two clones with homologous recombination were identified and isolated. ES cells from the two independent plexin-A1 mutant clones were injected separately into blastocysts from C57BL/6 mice. The blastocysts were transferred to pseudopregnant ICR foster mothers and chimeric males were then backcrossed to C57BL/6 or BALB/c females. Heterozygous mice were mated to produce homozygotes. For immunological analysis, heterozygous male mice were backcrossed to C57BL/6 or BALB/c females for five generations. Germline transmission and the genotype of plexin-A1-targeted allele were further assayed by Southern blot and PCR analysis. PCR was carried out with 35 cycles at 94° C. for 30 s, 60° C. for 30 s, 72° C. for 60 s. The following oligonucleotide primers were used to identify the rearranged plexin-A1 locus. Primer 1 (5′-AGCACCACACTCACACCCTCTTT-3′) was complementary to genomic DNA that was located in the 3′-untranslated region. Primer 2 (5′-TCCTTGATTTTCTCCTTGATGGCC-3′) was complementary to sequences at the 3′-terminus of the second exon. Primer 3 (5′-TCCCTGTCAGAGAAAACCTGGTTT-3′) was complementary to genomic DNA that was located in the untranslated region in the third exon. For Southern blot analysis, genomic DNA from the tails was digested with BamHI and subjected to agarose gel electrophoresis. DNA was transferred onto nylon blotting membranes (Hybond N; Amersham Pharmacia), according to the manufacturer's protocol. Filters were hybridized with radio-labelled probes overnight. Filters were then washed in 0.1×SSC, 0.1% SDS at 65° C. for one hour before autoradiography. For RT-PCR analysis, RNA was isolated from the brain, heart and spleen using RNeasy kits (Qiagen) and treated with DNase I (Invitrogen) to eliminate genomic DNA. cDNA was synthesized using a SuperScript II cDNA synthesis kit (Invitrogen) and RT-PCR was performed with 35 cycles at 94° C. for 30 s, 60° C. for 30 s, 72° C. for 30 s using the primers (5′-ACATCTACTATGTGTACAGTTTCC-3′) and (5′-AAAAACCACGGTGCGGCCTTGGGTA-3′). For northern blot analysis, total RNA isolated from the brain was subjected to formaldehyde-containing gel electrophoresis and transferred onto the blotting membrane and hybridized with radio-labelled probes overnight. Mice deficient in DAP12 were described previously 24. OT-2 Tg mice were kindly provided by Dr. William R. Heath 30. Mice were maintained in a specific pathogen-free environment. All experimental procedures were consistent with our institutional guidelines.

In vitro Assay

Splenic DCs were isolated from the spleen using MACS (Miltenyi Biotech). The resulting purity was >95% in each experiment. Bone marrow-derived DCs (BMDCs) were generated from bone marrow progenitors using GM-CSF. For FITC-dextran uptake, BMDCs were stained with allophycocyanin (APC)-conjugated anti-CD11c and incubated with pre-warmed medium containing 2 mg ml-1 FITC-dextran for 10 min at 37° C. After washing 3 times with chilled medium, internalised FITC-dextran was measured by FACS. For MLRs, irradiated (3000 rad) splenic DCs were cultured with allogeneic CD4+ T-cells (5×104 cells/well) for 48 h. To measure cell proliferation, cells were pulsed with 2 μCi of [3H] thymidine for the last 14 h of the culture period. In vivo T-cell responses For T-cell priming, mice were immunized with 100 μg of KLH in CFA into the hind footpads 7,8. Five days after immunization, CD4+ T-cells isolated from the draining lymph nodes were stimulated with various concentrations of KLH for 72 h. For proliferation assays, cells were pulsed with 2 μCi [3H] thymidine for the last 14 h. Cytokine production in the culture supernatants was measured by Bio-Plex suspension array system.

Osteoclast and Osteoblast Cultures

In vitro osteoclastogenesis was performed as described previously^{24,25}. In brief, bone marrow progenitor cells derived from wild-type- or plexin-A1−/− mice were cultured with M-CSF (10 ng ml-1) in α-MEM containing 10% FCS at 5×105 cells ml-1. At day 2, cells were harvested and further cultured for 3 days with M-CSF (10 ng ml-1) and RANKL (10 ng ml-1) at 5×104 cells ml-1 in flat-bottomed 96-well plates. The resulting cells were fixed and stained with tartrate-resistant acid phosphatase using a TRAP-staining kit (Takara, Japan). Primary osteoblasts were isolated from neonatal mouse calvaria after sequential digestion with 0.1% collagenase and 0.2% dispase. In co-culture experiments, calvarial osteoblasts and stromal cells were co-cultured with nonadherent bone marrow cells in medium supplemented with 10 nM 1.25(OH)2-vitamin D3 and 1 Mm prostaglandin E2.

Analysis of Bone Phenotype

Histological, histomorphmetric and microradiographic examinations were performed using essentially the same method as described previously²⁵. Statistical analysis was performed using Student's t-test (*p<0.05; **p<0.01; ***p<0.001).

Establishment of Stable Transfectants

Stable plexin-A1-, Trem-2-, and DAP12-expressing 293T cell transfectants were established by introducing Flag-tagged plexin-A1, V5-tagged Trem-2, and myc-tagged DAP12 expression constructs with pMC1neo vector by Lipofectamine (Invitrogen) according to the manufacturer's protocol. Transfectants expressing Flag-tagged plexin-A1, V5-tagged Trem-2 and myc-tagged DAP12 were selected in the presence of G418 and screened by anti-Flag mAb (M2, Sigma), anti-V5 mAb (Invitrogen) and anti-myc antibodies (9B11, Cell Signaling Technology) and cloned.

RNAi

Four siRNA sequences specific for mouse Trem-2 (5′-CCACGGTGCTGCAGGGCAT-3′, 5′-TGACCAAGATGCTGGAGAT-3′, 5′-CGGAATGGGAGCACAGTCA-3′ and 5′-GCACAGTCATCGCAGATGA-3′), were selected (Dharmacon). All siRNA sequences were synthesized and annealed by the manufacturer (Dharmacon). Transfection was performed using RNAiFect (QIAGEN) according to the manufacturer's protocol. Briefly, DCs were washed and plated in 24-well plates in complete RPMI 1640. siRNA were incubated with RNAiFect reagent in complete RPMI 1640 at room temperature for 10 min and then added to the DC culture. After 48 h of incubation, the resulting cells were harvested, washed and used for subsequent experiments. Transfection efficiencies were determined using fluorescein-labelled non-silencing RNA (40 to 50%).

Immunoprecipitation

Mouse antisera against mouse plexin-A1 were obtained by immunizing plexin-A1−/− mice with soluble plexin-A1 protein in CFA and used for immunoblotting. Rabbit antisera against mouse plexin-A1 were used for immunoprecipitation. Wild-type or DAP12−/− BMDCs were stimulated with anti-CD40 mAb for 24 h. Cells were solubilized in buffer containing 1% Digitonin, 10 mM Tris-Cl, 150 mM NaCl, 0.5 mM PMSF, 5 μg ml-1 aprotinin, 5 μg ml-1 leupeptin, and protease inhibitor cocktail (Nakarai, Japan). Cell lysates were incubated with protein A-sepharose plus anti-plexin-A1 for 3 h at 4° C. After washing five times with lysis buffer, immunoprecipitates were separated by SDS-PAGE and immunoblotted with anti-DAP12 25. Whole cell lysates were immunoblotted with anti-plexin-A1.

Phosphorylation of DAP12

RAW264.7 cells were co-transfected with Flag-tagged plexin-A1, V5-tagged Trem-2 and myc-tagged DAP12 expression constructs by Lipofectamine (Invitrogen) according to the manufacturer's protocol and incubated for 24 h. Cells were stimulated with 15 μg ml-1 Sema6D-Fc after 6 h of serum starvation. At various time points, cells were solubilized in buffer containing 1% Nonidet-P40, 10 mM Tris-Cl, 150 mM NaCl, 1 mM EDTA, 10 mM Na3VO4, 0.5 mM PMSF, 5 μg ml-1 aprotinin, 5 μg ml-1 leupeptin, and protease inhibitor cocktail (Roche). Cell lysates were incubated with protein G-agarose plus anti-myc mAb for 3 h at 4° C. After washing five times with lysis buffer, immunoprecipitates were separated by SDS-PAGE and immunoblotted with anti-phosphotyrosine (4G10, Upstate Biotechnology) or anti-myc Abs.

### REFERENCES

All publications cited in this application are hereby incorporated by reference in their entirety.

1. Tessier-Lavigne, M. & Goodman, S. C. The molecular biology of axon guidance. Science 274, 1123-1133 (1996).

2. Pasterkamp, R. J. & Kolodkin, A. L. Semaphorin junction: making tracks toward neural connectivity. Curr Opin Neurobiol 13, 79-89 (2003).

3. Sekido, Y. et al. Human semaphorins A(V) and IV reside in the 3p21.3 small cell lung cancer deletion region and demonstrate distinct expression patterns. Proc Natl Acad Sci USA 93, 4120-5 (1996).

4. Gu, C. et al. Neuropilin-1 conveys semaphorin and VEGF signaling during neural and cardiovascular development. Dev Cell 5, 45-57 (2003).

5. Toyofuku, T. et al. Guidance of myocardial patterning in cardiac development by Sema6D reverse signalling. Nat Cell Biol 6, 1204-11 (2004).

6. Kumanogoh, A. et al. Identification of CD72 as a lymphocyte receptor for the class IV semaphorin CD100: a novel mechanism for regulating B cell signaling. Immunity 13, 621-31. (2000).

7. Shi, W. et al. The class IV semaphorin CD100 plays nonredundant roles in the immune system: defective B and T cell activation in CD100-deficient mice. Immunity 13, 633-42. (2000).

8. Kumanogoh, A. et al. Class IV semaphorin Sema4A enhances T-cell activation and interacts with Tim-2. Nature 419, 629-33 (2002).

9. Kumanogoh, A. et al. Nonredundant Roles of Sema4A in the Immune System: Defective T Cell Priming and Th1/Th2 Regulation in Sema4A-Deficient Mice. Immunity 22, 305-16 (2005).

10. Kikutani, H. & Kumanogoh, A. Semaphorins in interactions between T cells and antigen-presenting cells. Nat Rev Immunol 3, 159-67 (2003).

11. Elhabazi, A., Marie-Cardine, A., Chabbert-de Ponnat, I., Bensussan, A. & Boumsell, L. Structure and function of the immune semaphoring CD100/SEMA4D. Crit Rev Immunol 23, 65-81 (2003).

12. Tamagnone, L. & Comoglio, P. M. Signalling by semaphorin receptors: cell guidance and beyond. Trends Cell Biology 10, 377-383 (2000).

13. Granziero, L. et al. CD100/Plexin-B1 interactions sustain proliferation and survival of normal and leukemic CD5+ B lymphocytes. Blood 101, 1962-9 (2003).

14. Walzer, T., Galibert, L., Comeau, M. R. & De Smedt, T. Plexin C1 engagement on mouse dendritic cells by viral semaphorin A39R induces actin cytoskeleton rearrangement and inhibits integrin-mediated adhesion and chemokine-induced migration. J Immunol 174, 51-9 (2005).

15. Takahashi, T. et al. Plexin-neuropilin-1 complexes form functional semaphorin-3A receptors. Cell 99, 59-69. (1999).

16. Toyofuku, T. et al. Dual roles of Sema6D in cardiac morphogenesis through region-specific association of its receptor, Plexin-A1, with off-track and vascular endothelial growth factor receptor type 2. Genes Dev 18, 435-47 (2004).

17. Wong, A. W. et al. CIITA-regulated plexin-A1 affects T-cell-dendritic cell interactions. Nat Immunol 4, 891-8 (2003).

18. Lacey, D. L. et al. Osteoprotegerin ligand is a cytokine that regulates osteoclastdifferentiation and activation. Cell 93, 165-76 (1998).

19. Theill, L. E., Boyle, W. J. & Penninger, J. M. RANK-L and RANK: T cells, bone loss, and mammalian evolution. Annu Rev Immunol 20, 795-823 (2002).

20. Takahashi, T. & Strittmatter, S. M. Plexinal autoinhibition by the plexin sema domain. Neuron 29, 429-39. (2001).

21. Giordano, S. et al. The semaphorin 4D receptor controls invasive growth by coupling with Met. Nat Cell Biol 4, 720-4 (2002).

22. Lanier, L. L. & Bakker, A. B. The ITAM-bearing transmembrane adaptor DAP12 in lymphoid and myeloid cell function. Immunol Today 21, 611-4 (2000).

23. Colonna, M. TREMs in the immune system and beyond. Nat Rev Immunol 3,445-53 (2003).

24. Kaifu, T. et al. Osteopetrosis and thalamic hypomyelinosis with synaptic degeneration in DAP12-deficient mice. J Clin Invest 111, 323-32 (2003).

25. Koga, T. et al. Costimulatory signals mediated by the ITAM motif cooperate with RANKL for bone homeostasis. Nature 428, 758-63 (2004).

26. Bakker, A. B. et al. DAP12-deficient mice fail to develop autoimmunity due to impaired antigen priming. Immunity 13, 345-53 (2000).

27. Paloneva, J. et al. Loss-of-function mutations in TYROBP (DAP 12) result in a presenile dementia with bone cysts. Nat Genet 25, 357-61 (2000).

28. Paloneva, J. et al. Mutations in two genes encoding different subunits of a receptor signaling complex result in an identical disease phenotype. Am J Hum Genet 71, 656-62 (2002).

29. Turner, L. J., Nicholls, S. & Hall, A. The activity of the plexin-A1 receptor is regulated by Rac. J Biol Chem 279, 33199-205 (2004).

30. Barnden, M. J., Allison, J., Heath, W. R. & Carbone, F. R. Defective TCR expression in transgenic mice constructed using cDNA-based alpha- and 21 beta-chain genes under the control of heterologous regulatory elements. Immunol Cell Biol 76, 34-40 (1998).

1. A method of treating an inflammatory, autoimmune or bone resorption disease, the method comprising administering to a patient a therapeutically effective amount of a composition which inhibits plexin-A1-DAP12 interaction.

2. A method to identify a compound that inhibits interaction of plexin-A1 with DAP12 activity in a cell, comprising: (1) contacting a cell with a putative regulatory compound, wherein the cell includes a plexin-A1 protein and a DAP12 protein; and (2) assessing the ability of the putative regulatory compound to inhibit the interaction of plexin-A1 with DAP12.

3. A method to identify a compound that inhibits interaction of plexin-A1 with Trem-2 activity in a cell, comprising: (1) contacting a cell with a putative regulatory compound, wherein the cell includes a plexin-A1 protein and a Trem-2 protein; and (2) assessing the ability of the putative regulatory compound to inhibit the interaction of plexin-A1 with Trem-2.

4. A method to identify a compound that inhibits interaction of DAP12 with Trem-2 activity in a cell, comprising: (1) contacting a cell with a putative regulatory compound, wherein the cell includes a DAP12 protein and a Trem-2 protein; and (2) assessing the ability of the putative regulatory compound to inhibit the interaction of DAP12 with Trem-2.

5. An antibody or antibody binding site which effectively binds plexin-A1, Trem-2 or DAP12 or fragments thereof, wherein the above antibody or antibody binding site which binds plexin-A1, Trem-2 or DAP12 inhibits binding of plexin-A1 to DAP12, plexin-A1 binding to Trem-2, or Trem-2 binding to DAP12.

6. A biotherapeutic composition for treating an inflammatory, autoimmune or bone resorption disease comprising a therapeutically effective amount of plexin-A1 protein, Trem-2 protein or DAP12 protein or fragments thereof.
